(19) United States
(12) Patent Application Publication
(10) Pub. No.: US 2015/0125401 A1
(43) Pub. Date: May 7, 2015

(54) SMALL MAGNETITE THERAPEUTICS AND METHODS OF USE THEREOF

(71) Applicants: BOARD OF THE REGENTS OF THE UNIVERSITY OF NEBRASKA, LIncoln, NE (US); VIRGINIA TECH INTELLECTUAL PROPERTIES, INC., Blacksburg, VA (US)

(72) Inventors: Howard E. Gendelman, Omaha, NE (US); Alexander V. Kabanov, Chapel Hill, NC (US); Xin-Ming Liu, Omaha, NE (US); Richey M. Davis, Blacksburg, VA (US); Judy Riffle, Blacksburg, VA (US)

(21) Appl. No.: 14/394,037

(22) PCT Filed: Apr. 15, 2013
(86) PCT No.: PCT/US2013/036594

§ 371 Date: Oct. 10, 2014

Related U.S. Application Data
(60) Provisional application No. 61/636,042, filed on Apr. 20, 2012.

(51) Int. Cl. A61K 41/00; A61K 49/18; A61K 45/06; A61K 9/50; A61K 31/427

(52) U.S. Cl. CPC A61K 41/00; A61K 9/5031; A61K 31/427; A61K 45/06; A61K 49/186
USPC 424/9.322; 424/497; 514/365

(57) ABSTRACT

The present invention provides compositions and methods for the delivery of therapeutics to a cell or subject.

This application claims priority under 35 U.S.C. §119(e) to U.S. Provisional Patent Application No. 61/636,042, filed Apr. 20, 2012. The foregoing application is incorporated by reference herein.

This invention was made with government support under Grant No. 1P01 DA028555 awarded by the National Institutes of Health and Grant No. DMR-0909065 awarded by the National Science Foundation. The government has certain rights in the invention.

### FIELD OF THE INVENTION

The present invention relates generally to the delivery of therapeutic and diagnostic agents. More specifically, the present invention relates to compositions and methods for the delivery of therapeutic and diagnostic agents to a patient, particularly for the treatment of a microbial infection, particularly a viral infection.

### BACKGROUND OF THE INVENTION

Combination antiretroviral (cART), now administered over decades to human immunodeficiency virus (HIV) infected people, can lead to cardiovascular, neoplastic, liver, kidney, bone and immune disorders (Corbett et al. (2002) Ann. Pharmacother., 36:1193-203; Hruz, P. W. (2011) Best Pract. Res. Clin. Endocrinol. Metab., 25:459-68; Veloso et al. (2010) Curr. Pharm. Des., 30:3379-89; Domingo, P. (2009) Enferm. Infecc. Microbiol. Clin., 27 Suppl 2:46-51). The antiretroviral therapy can accelerate cognitive impairment, systemic diseases and aging (Effros et al. (2008) Clin. Infect. Dis., 47:542-53). Accordingly, it is clear that the toxicities associated with antiretroviral therapy are undesirably high with current methods. Further, eradication of HIV in its infected human host requires antiretroviral drug delivery to viral sanctuaries with the secondary elimination of latent or restricted infections (Wainberg, M. A. (2011) Nature 469:306-307). The former could be facilitated through targeted nanoparticle drug delivery but, to achieve its potential, would require improved virus-target tissue drug bioavailability. One major hurdle towards achieving this goal is the dearth of any means to measure antiretroviral therapy (ART) distribution outside of plasma drug levels (Pretorius et al. (2011) Ther. Drug Monit., 33:265-274). In view of the foregoing, there is a clear need for improved drug delivery systems.

### SUMMARY OF THE INVENTION

In accordance with the instant invention, nanoparticles comprising at least one therapeutic agent, at least one amphiphilic compound, and at least one paramagnetic particle are provided. In a particular embodiment, the amphiphilic compound is an amphiphilic block copolymer, phospholipid, and/or PEGylated phospholipid. In a particular embodiment, the amphiphilic compound is linked to at least one targeting ligand such as a macrophage targeting ligand. In a particular embodiment, the therapeutic agent is an antimicrobial (e.g., an antibacterial, an antiviral, antiretroviral, or anti-HIV compound). Compositions comprising at least nanoparticle of the instant invention and at least one pharmaceutically acceptable carrier are also provided. Methods of synthesizing the nanoparticle of the instant invention are also provided.

According to another aspect of the instant invention, methods for monitoring therapeutic agents distribution and methods for treating, inhibiting, or preventing a disease or disorder in a subject are provided. In a particular embodiment, the method comprises administering to the subject at least one nanoparticle of the instant invention. In a particular embodiment, the methods are for treating, inhibiting, or preventing an HIV infection and the therapeutic agent of the nanoparticle is an anti-HIV compound. In a particular embodiment, the method further comprises administering at least one further therapeutic agent or therapy for the disease or disorder, e.g., at least one additional anti-HIV compound.

### BRIEF DESCRIPTIONS OF THE DRAWING

FIG. 1A provides a schematic of the structure of lipid-coated polylactic-co-glycolic acid (PLGA), small magnetite antiretroviral therapeutic (SMART). Atazanavir (ATV) and superparamagnetic iron oxide particles (SPIOs, (e.g., ultrasmall superparamagnetic iron oxide particles (USPIOs)) are well distributed into the PLGA matrix to form the core of SMART. The PLGA core is coated with lipid monolayer to form the shell of SMART. FIG. 1B provides a representative transmission electron micrograph (TEM) of a single SMART particle. FIG. 1C provides a timecourse of uptake (upper panel) and retention (lower panel) of SMART in monocyte-derived macrophages (MDM). MDM were treated with 100 μM SMART (based on ATV content) for 1, 2, 4 and 8 hour uptake studies. After treated MDM with 100 μM SMART for 8 hours, cell culture media were changed for 0, 5, 10, 15 day retention studies. The cell lysates at indicated time points were analyzed by HPLC and ICP-MS for ATV and magnetite quantification. Data represent the mean±SEM, n=3 for each time point. FIG. 1D provides images of Prussian blue stain of MDM. MDM were treated with SMART in PBS (lower panel) and PBS (negative control, upper panel) for 24 hours and then fixed with 2% formalin/2.5% glutaraldehyde in PBS and stained with 5% potassium ferrocyanide/5% hydrochloric acid (1:1).

FIG. 2 provides graphs of the concentration dependence of relaxivity (r₂) of SMART in PBS (FIG. 2A) and MDM (FIG. 2B). MDM were incubated with 100 μM SMART (based on ATV content) for 24 hours. Collected MDM and SMART were suspended in 1% agar gel. T₂ was measured by magnetic resonance imaging (MRI), and magnetite content by inductively coupled mass spectrometry (ICP-MS).

FIG. 3 provides MRI assessments of the tissue drug biodistribution and pharmocokinetics by SMART particles. After pre-MRI scan, mice were injected with SMART through a jugular vein cannula, and then scanned by MRI at continuously at 30 minute intervals up to 4 hours after SMART administration. Mean tissue SMART content was determined Immediately after the final scan, mice were euthanized and tissues were collected for ATV quantification by ultra-performance liquid chromatography tandem mass-spectrometry (UPLC-MS/MS). FIG. 3A provides an MRI based images of magnetite concentration in kidney, spleen and liver from 0.5 hour to 4 hours following SMART administration. FIG. 3B provides a graph of magnetite (per mg iron) levels in kidney, spleen and liver over 4 hours following SMART administration.

FIG. 4 provides 3D gradient recalled echo images of the same mouse before (FIG. 4A) and 4 hours after (FIG. 4B) injection of SMART. The signal from the liver is completely eliminated due to the accumulation of magnetite loaded SMART (L=lung, Lv=liver, K=kidney and S=spleen).

FIG. 5 provides a graph of the correlation of SMART-associated magnetite and ATV in tissues 24 hours after administration. The magnetite concentration was quantified from the change in T₂ weighted relaxivity (ΔR₂=1/T_{2preinjection}−1/T_{2postinjection}) and the per milligram magnetite relaxivity (r₂) determined as the slope of magnetite concentration versus R₂ in SMART phantom studies. ATV concentrations were quantified by UPLC-MS/MS following the final 24 hour MRI scan.

FIG. 6 provides immunohistology of ionized calcium binding adapter molecule 1 (Iba-1) staining and Prussian blue staining of liver with Prussian blue (FIG. 6A; 200×), liver with Prussian blue and IBA-1 (FIG. 6B; 200×), enlargement from FIG. 6B (FIG. 6C), spleen with Prussian blue (FIG. 6D; 200×), spleen with Prussian blue and IBA-1 (FIG. 6E; 200×), enlargement from FIG. 6E (FIG. 6F). Livers and spleens were fixed with 10% formalin, paraffin embedded and sectioned for immunohistological analysis after the final MRI scan. Macrophages were identified by Iba1 stains and magnetite identified by Prussian blue. Fl=splenic follicle, M=splenic mesentery.

FIG. 7 provides a schematic of the reaction of a cysteine amino acid and a maleimide functionalized polymer.

### DETAILED DESCRIPTION OF THE INVENTION

The instant invention provides combinations of small magnetite particles and antiretroviral therapeutics (ART) in a single nanoparticle. Such small magnetite ART (SMART) permits rapid pharmacokinetic and biodistribution evaluations of ART in virus-target tissues, such as the lymph nodes and brain. Drug biodistribution can be readily quantitated, such as by a conventional magnetic resonance imaging (MRI) scan. This approach also provides the ability to deliver packaged medicines to sites of limited viral growth and serve, at least in part, to eliminate the viral reservoir. Magnetically targeted cancer drug delivery utilizing T₂- or T₂*- has been quantified by MRI (Girard et al. (2012) Contrast Med. Mol. Imaging 7:411-417; Guthi et al. (2010) Mol. Pharm., 7:32-40; Lebel et al. (2006) Magn. Res. Med., 55:583-591; Liu et al. (2009) Magn. Res. Med., 61:761-766).

Herein, magnetite (also referred to as superparamagnetic iron oxide particles (SPIOs) or USPIOs) was inserted into lipid-coated polylactic-co-glycolic acid (PLGA) nanoparticles with a commonly used antiretroviral protease inhibitor, atazanavir (ATV), as a particular example for the instant theranostic approach. By combining PLGA and magnetite, organic/inorganic hybrid composite biomaterials allowed combined diagnostics, or drug distribution assessments, with therapeutic ART delivery through a single MRI scan (Kabanov et al. (2007) Prog. Polym. Sci., 32:1054-1082). The SMART nanoparticle testing was sped through the availability of in vitro cultivated monocyte-derived macrophages (MDM) that determined optimal particle cell uptake and retention. This facilitated studies of the dynamics of in vivo drug tissue distribution. The results presented herein demonstrate the utility of SMART systems for noninvasive drug pharmacokinetics for the inevitable goal of viral eradication.

To allow for the rapid noninvasive determination of drug biodistribution in virus-target tissues and reservoirs for therapeutics such as nanoART, the instant invention provides small magnetite ART (SMART) particles which allow for noninvasive assessments of antiretroviral drug pharmokinetics and tissue distribution through MRI techniques. Specifically, poly(lactic-co-glycolic acid), 1,2-distearoyl-snglycero-3-phosphocholine and 1,2-distearoyl-sn-glycero-3-phosphoethanolamine-N-[methoxy (polyethylene glycol)-2000] encased particles were synthesized that contained ATV and magnetite. Cellular uptake and retention of magnetite and ATV were first performed in human MDM. Tandem mass spectrometry showed that SMART particles were efficiently taken up and retained in MDM. In mice, magnetite and drug biodistribution, paralleled one another, as readily seen after parenteral injections. Three to one ratios of ATV to magnetite allowed drug assessments, for proof of concept experiments, at 4 to 24 hours after particle injection. T₂ maps and 3D spoiled gradient recalled echo image sets confirmed rapid drug tissue distribution in the reticuloendothelial system including spleen, liver, kidney and lung. At four hours, T₂ mapping showed predominant vascular particle distribution. However, by 24 hours signal intensity was seen in liver and spleen with little to no magnetite in kidneys. Significantly, ATV tissue levels correlated with changes in tissue relaxivity (ΔR₂=1 /T_{2postinjection}−1/T_{2preinjection}). Thus, SMART can facilitate the evaluation of drug tissue concentrations in viral reservoirs and provide rapid assessments for the next generation cell and tissue ligand decorated particles.

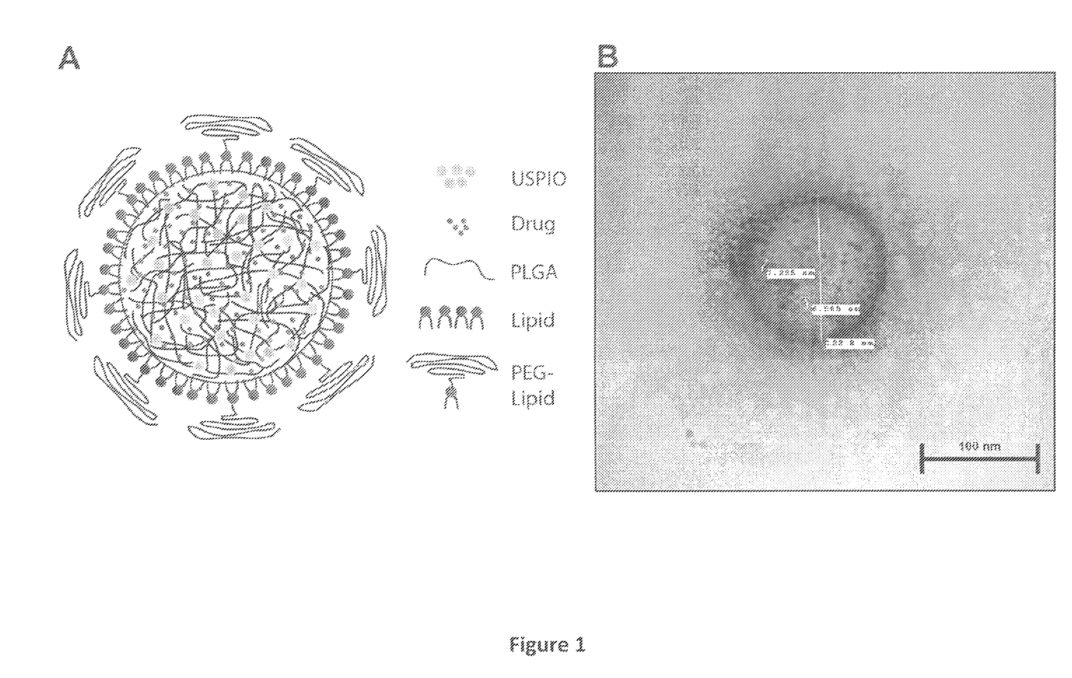

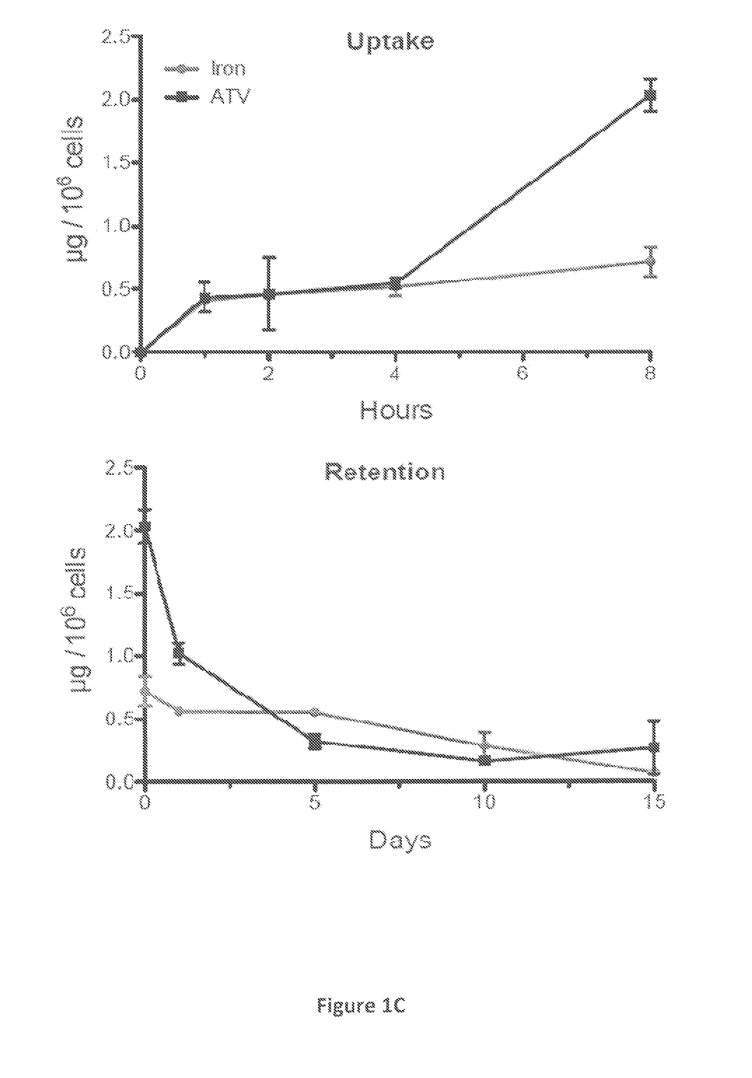

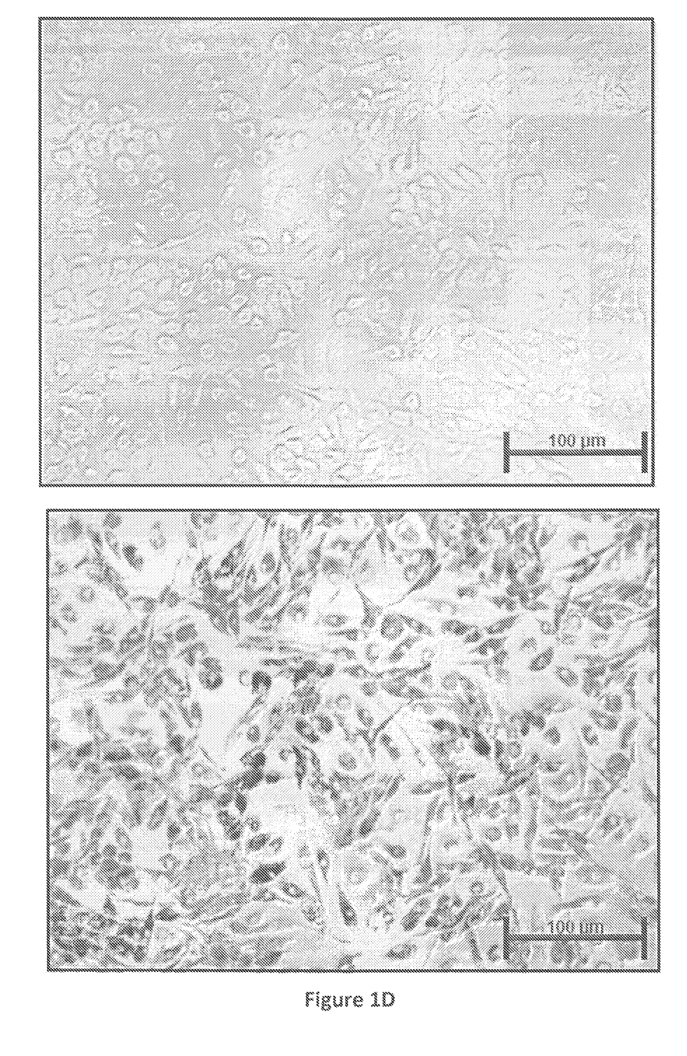

As seen in FIG. 1A, the nanoparticles of the instant invention comprise a hydrophobic core. The hydrophobic core comprises superparamagnetic iron oxide particles. The hydrophobic core may also comprise at least one therapeutic agent, such as antiretroviral therapeutics (ART), and/or at least one imaging agent. Indeed, combinations of multiple drugs and/or drugs with imaging agents may be encapsulated into a single nanoparticle. The nanoparticles may also comprise an outer shell surrounding the hydrophobic core. The outer shell may be hydrophilic and allow for steric stability of the nanoparticle. In a particular embodiment, the outer shell comprises an amphiphilic compound such as an amphiphilic block copolymer, phospholipid, and/or PEGylated phospholipid. For example, the hydrophilic block(s) of the amphiphilic block copolymer may be poly(ethylene oxide). In a particular embodiment, the amphiphilic block copolymer comprises polyanhydride, polyester, poly(propylene oxide), poly(1,2-butylene oxide), poly(n-butylene oxide), poly(tetrahydrofurane), or poly(styrene) as a hydrophobic block. The components of the nanoparticle, along with other optional components, are described in more detail hereinbelow.

The nanoparticles of the instant invention can be used for noninvasive real-time assessment of drug biodistribution (e.g., for personalized medicine). These nanoparticles will aide in developing optimal formulations for each patient that enable viral clearance and reduce ART toxicities in infected people. Additionally, the nanoparticles can be used for drug-drug and imaging agent-drug combinations to treat the infection and/or monitor drug distribution within a patient.

For manufacturing of the nanoparticles it is envisioned that numerous methods can be used. Indeed, the nanoparticles of this invention may be prepared by various methods including but not limited to homogenization, wet-milling, sonication, single emulsion, double emulsion, and flash nanoprecipitation. In a particular embodiment, flash precipitation is used to manufacture the nanoparticles. This process is reproducible and scalable with high drug loading capacity. Indeed, flash precipitation allows for narrow particle size distributions and improved bioavailability of drug encased polymers. Methods of flash precipitation are known in the art (e.g., Liu et al. (2008) Chem. Engr. Sci., 63:2829-2842; Gindy et al. (2008) Langmuir 24:83-90; U.S. Pat. No. 8,137,699; U.S. Patent Application Publication No. 2010/0330368). Briefly, a vortex mixer, particularly a multi-inlet vortex mixer (MIVM; e.g., a 4-jet multi-inlet vortex mixer) may be used to rapidly combine the organic solution of amphiphilic compound (e.g., amphiphilic block copolymer), compound to be encapsulated (e.g., therapeutic agent (e.g., ART)), hydrophobically modified magnetite nanoparticles, and other components with water or buffer. This rapid mixing creates high supersaturations of drug and magnetite leading to SMART nucleation, whereby size is controlled by copolymer self-assembly. These nanoparticles contain hydrophobic cores encapsulated with both ART and magnetite and are surrounded by a corona of hydrophilic polymer (e.g., PEO chains) for steric stability. This process is reproducible and scalable with high drug loading capacity.

Nanoparticle uptake by cells and biodistribution depends on size, shape, and surface chemistry (Doshi et al. (2009) Adv. Funct. Matr., 19:3843-54; Doshi et al. (2009) PNAS, 106:21495-9; Euliss et al. (2006) Chem. Soc. Rev., 35:1095-104; Rolland et al. (2005) JACS, 127:10096-100; Gratton et al. (2008) Pharm. Res., 25:2845-52; Gratton et al. (2008) PNAS, 105:11613-8). Controlling the corona chemistry and particle size of the instant nanoparticles will help determine particle uptake by MDMs. Further, controlling the polymer chemistry in the core will help determine ART and magnetite loading in the particles. ART release kinetics from the particles will also be mainly controlled by the particle size (through the surface/volume ratio) and the polymer chemistry in the particle cores. The nanoparticles of the instant invention provide flexibility to allow for changes in particle size, polymer corona chemistry, and core chemistry.

Flash nanoprecipitation can be utilized to make particles with narrow size distributions in the range of about 30-500 nm. As initially demonstrated hereinbelow, particle sizes can be controlled by varying the compositions and molecular weights of the polymers, the degree of supersaturation of drugs, and magnetite loading in the multi-inlet vortex mixer (MIVM). Hydrophobic polymers (e.g., homopolymers) such as PCL can serve as nucleation agents for therapeutics such as ART drugs while co-precipitating them with amphiphilic copolymers which stabilize the particles (D'Addio et al. (2011) Adv. Drug Deliv. Rev., 63:417-26). Polymers such as PCL, PLGA, and PLLA may be used for this purpose when using, e.g., PCL-PEO, PLGA-PEO, poloxamers, and PDLLA-PEO as diblock stabilizers.

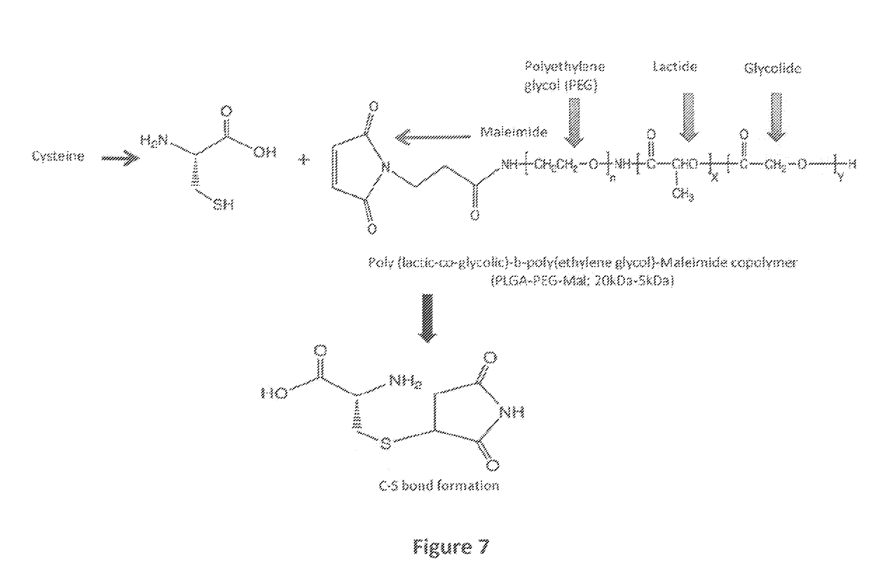

The nanoparticles of the instant invention may also comprise amphiphilic compounds such as phospholipids, PEGylated phospholipids, and/or amphiphilic block copolymers (e.g., PCL-PEO diblocks) where the hydrophilic block (e.g., PEO chain) is terminated with a functional group (e.g., amine, carboxy, cysteine, azide, acetylene group, etc.) to allow attachment of a compound to the polymer. In particular embodiment, the functional group is a maleimide group. Maleimide groups may be readily conjugated with a protein (Gindy et al. (2008) Biomacromolecules 9:2705-11). FIG. 7 provides a schematic of the chemical reaction for attaching a polypeptide to a maleimide of a polymer, thereby forming a covalent attachment (i.e. a C—S bond). Indeed, the nanoparticles of then instant invention may comprise a targeting group (e.g., folic acid, polypeptide, polysaccharide, and/or sugar) covalently attached to the nanoparticle.

In addition, copolymers containing varying sizes and/or amounts of hydrophobic blocks such as poly(propylene oxide) (PPO) may be used in the synthesis of the nanoparticles because varying the hydrophilic/hydrophobic balance of polymers in the coronas surrounding the polyester cores can affect particle uptake (Batrakova et al. (2010) J. Controlled Rel., 143:290-301; Sahay et al. (2010) Biomaterials 31:923-33; Kabanov et al. (2005) J. Controlled Rel., 101:259-71). For example, the hydrophilic/hydrophobic balance may be varied by co-precipitating PPO-containing block copolymers along with PEO-containing block copolymers in the MIVM. Examples of PPO-containing block copolymers include, without limitation, Pluronic® polymers including pentablock copolymers comprising a Pluronic® polymer and Pluronic® polymers comprising targeting endgroups (e.g., folic acid, peptides, and sugars). Examples of pentablock copolymers include, without limitation, PCL-Pluronic®-PCL and PLLA-Pluronic®-PLLA. These pentablock copolymers can be co-precipitated with PCL-PEO and PDLLA-PEO diblocks, respectively, to obtain particles with tailored PPO/PEO ratios. As shown hereinbelow, colloidally stable PCL-based particles have been synthesized with PPO/PEO wt/wt ratios ranging from 0.14-0.33 and with ATV loadings as high as 30 wt % and, separately, magnetite loadings as high as 16 wt %. In work with nanoART, good particle uptake by MDMs occurred when the zeta potential of the particles was as high as −40 mV (Nowacek et al. (2011) J. Control Rel., 150:204-11). The zeta potential of the particles of the instant invention made by flash nanoprecipitation was approximately −5 mV, which is consistent with nanoparticles that are stabilized primarily by steric repulsions between PEO coronas. To tune the zeta potential, pentablock copolymers consisting of polyacrylic acid (PAA) blocks covalently coupled to both ends of Pluronic® triblocks, denoted as PAA-Pluronic®-PAA can be incorporated into the nanoparticles. These may be co-precipitated along with the PDLLA- and PCL-based copolymers at various weight ratios to tune the zeta potential from ˜0 to −40 mV.

To facilitate cell uptake and intracellular trafficking studies using confocal microscopy, imaging agents such as fluorophores may be incorporated into the SMART particles. For example, rhodamine dye could be covalently coupled to the PAA-Pluronics-PAA pentablocks and these could be co-precipitated with the PDLLA- and PCL-based copolymers.

In addition to particle size and zeta potential, particle uptake by cells is affected by the binding of proteins to particles (Li et al. (2009) Biochim. Biophys. Acta., 1788:2259-66; Tenzer et al. (2011) Acs Nano 5:7155-67). The nanoparticles of the instant invention may have coronas (e.g., PEO coronas) sufficiently dense to prevent nonspecific protein adsorption and comprise covalently attach functional moieties, including proteins, to control uptake and targeting. This reduces potentially irreproducible effects when proteins physisorb to nanoparticles, affecting how they interact with cells. Further, the method introduces a level of control that will enable the efficient regulation of particle uptake. It has been demonstrated that magnetite particles (Feridex™) that were conjugated with IgG showed ˜4× higher uptake by MDMs than unconjugated Feridex™ (Beduneau et al. (2009) PLoS One 4:e4343). Nanoparticles of PDLLA (137 k) homopolymer stabilized with PDLLA (30 k)-PEO (2 k) diblock copolymer (in a 1:1 wt:wt ratio) also showed that the PEO corona suppressed adsorption of plasma factors that trigger the coagulation cascade (Sahli et al. (1997) Biomaterials 18:281-8). Particle-protein binding can be characterized using, for example, nanoparticle tracking analysis (NTA). The size distributions of particles incubated with cell culture media containing proteins may be compared to the size distribution in PBS. Protein binding to the nanoparticles will result in a shift of the size distribution to larger sizes. In addition, it can be determined whether the nanoparticles activate the coagulation cascade and the complement system using a variety of techniques including cytokine arrays.

As shown hereinbelow, PPO-containing pentablock copolymers improve the compatibility of the semicrystalline PCL for ART drugs such as ATV. When PCL-Pluronic®-PCL pentablocks were blended with the diblock PCL-PEO, the ATV loading was 30 wt % for a targeted loading of 30 wt %, a 57% increase over the ATV loading in nanoparticles consisting of just the diblock PCL-PEO. This was found for two pentablocks: PCL-Pluronic® F68-PCL and PCL-Pluronic® P85-PCL. Hydrophobic blocks other than PPO can also be used.

In addition, more than one therapeutic agent may be loaded in to the nanoparticles of the instant invention. For example, combinations of ART (e.g., ATV with RTV) may be combined in a given nanoparticle system to enable combination ART therapy. The therapeutic agents may be present in various wt/wt ratios. For example, a target value for loading ATV and RTV together is an ATV/RTV wt/wt ratio=3/1.

Polyesters such as polylactic acids and polycaprolactone degrade primarily by bulk degradation in which water diffuses into the particle, leading to hydrolysis of the polymer backbone (Uhrich et al. (1999) Chem. Rev., 99:3181-98). PLLA degrades more slowly than PDLLA due to the crystallinity of the PLLA (Conti et al. (1992) J. Microencapsul., 9:153-66). The crystallinity and glass transition temperature (Tg) of the particles are, in general, functions of the particle composition and processing history. For nanoparticles made with PLLA/PDLLA-PEO and those made with PCL-PEO diblocks and PCL-Pluronic-PCL pentablock copolymers, there is a correlation between crystallinity and the particle degradation rates. The wt:wt ratios and molecular weights of the polymer blends can be varied to affect crystallinity and Tg (which can be measured using differential scanning calorimetry and X-ray diffraction). Degradation rates can be measured in vitro using, for example, particle size measurements obtained from dynamic light scattering and nanoparticle tracking analysis.

The drug release rate is a complex function of particle size, composition, polymer morphology, and the glass transition temperature (Uhrich et al. (1999) Chem. Rev., 99:3181-98; Vert et al. (1994) Biomaterials 15:1209-13; Kumar et al. (2009) Mol. Pharm., 6:1118-24). For a sphere with radius “R”, the (surface/volume)=3/R so reducing a particle diameter from 300 to 100 nm increases the (surface/volume) ratio by 300%. As another example, the diffusion rate of a drug through a glassy polymer matrix (T<Tg) can be orders of magnitude slower than that through a rubbery matrix (T>Tg).

The results presented herein indicate that the ART drugs and hydrophobically modified magnetite compete for space in the polyester cores of the nanoparticles. For a PDLLA (4 k)-PEO (5 k) diblock, particles were made with 38 wt % RTV loading. However, when magnetite nanoparticles (˜8 nm diameter) were loaded at up to 31 wt %, the RTV loading dropped to 7 wt %. Accordingly, one can vary the components to optimize the loading of magnetite to obtain a sufficiently high transverse relaxivity to enable biodistribution studies while also maximizing the ART drug loading. In a particular embodiment, the magnetite can be directly conjugated to the therapeutic agent.

The sensitivity of MRI distribution measurements will depend on the nanoparticles loading in the target cells and the transverse relaxivity (r₂) of the particles. The value of r₂ depends on the size and magnetite composition of the nanoparticles. Feridex™ magnetite contrast agent particles aggregated in intracellular compartments in MDMs (Beduneau et al. (2009) PLoS One 4:e4343). This can lead to higher effective r₂ values for the particles compared to those measured for the same particles dispersed in a buffer such as PBS. MRI measurements of cells that have internalized particles containing nanoparticles of the instant invention can be used to measure the average drug concentration in the cells.

As explained hereinabove, the instant invention encompasses nanoparticles for the delivery of compounds to a cell. In a particular embodiment, the nanoparticle is for the delivery of antiretroviral therapy to a subject. In a particular embodiment, the nanoparticle of the instant invention is up to 1 μm in diameter. In a particular embodiment, the nanoparticle is about 50 nm to about 500 nm in diameter, particularly about 100-500 nm, 100-250, or 100-150 nm in diameter. In a particular embodiment, the nanoparticles have a PDI of less than 0.20. The components of the nanoparticle, along with other optional components, are described in more detail hereinbelow.

### I. Encapsulated Agent

The nanoparticles of the instant invention may be used to deliver any agent(s) or compound(s), particularly bioactive agents (e.g., therapeutic agent or diagnostic/imaging agent) to a cell or a subject (including non-human animals). The encapsulated agent/compound can be hydrophobic and hydrophilic. As used herein, the term “bioactive agent” also includes compounds to be screened as potential leads in the development of drugs or plant protecting agents. Bioactive agent include, without limitation, polypeptides, peptides, glycoproteins, nucleic acids, synthetic and natural drugs, peptoides, polyenes, macrocyles, glycosides, terpenes, terpenoids, aliphatic and aromatic compounds, small molecules, and their derivatives and salts. In a particular embodiment, the therapeutic agent is a chemical compound such as a synthetic and natural drug. The nanoparticles of the instant invention may comprise one or more agent or compound. For example, the nanoparticles may comprise more than one therapeutic agent, more than one imaging agent, or one or more therapeutic agents with one or more imaging agent.

While any type of compound may be delivered to a cell or subject by the compositions and methods of the instant invention—as explained above, the following description of the inventions generally exemplifies the compound as a therapeutic agent for simplicity.

The agent/compound (e.g. therapeutic agent) may be hydrophilic, a water soluble compound, hydrophobic, a water insoluble compound, or a poorly water soluble compound. In a particular embodiment, the agent/compound is hydrophobic. For example, the therapeutic agent may have a solubility of less than about 10 mg/ml, less than 1 mg/ml, more particularly less than about 100 μg/ml, and more particularly less than about 25 μg/ml in water or aqueous media in a pH range of 0-14, particularly between pH 4 and 10, particularly at 20° C.

In a particular embodiment, the therapeutic agent of the nanoparticles of the instant invention is an antimicrobial (e.g., antibiotic/antibacterial (e.g., antituberculosis drugs)). In another embodiment, the therapeutic agent is an antiviral, more particularly an antiretroviral therapeutic. The antiretroviral may be effective against or specific to lentiviruses. Lentiviruses include, without limitation, human immunodeficiency virus (HIV) (e.g., HIV-1, HIV-2), bovine immunodeficiency virus (BIV), feline immunodeficiency virus (FIV), simian immunodeficiency virus (SIV), and equine infectious anemia virus (EIA). In a particular embodiment, the therapeutic agent is an anti-HIV agent. An anti-HIV compound or an anti-HIV agent is a compound which inhibits HIV. Examples of antiretroviral therapeutics (e.g., anti-HIV agents) include, without limitation:

(I) Nucleoside-analog reverse transcriptase inhibitors (NRTIs). NRTIs refer to nucleosides and nucleotides and analogues thereof that inhibit the activity of reverse transcriptase, particularly HIV-1 reverse transcriptase. An example of nucleoside-analog reverse transcriptase inhibitors is, without limitation, adefovir, adefovir dipivoxil, zidovudine (AZT, retrovir), didanosine (Videx, ddl), zalcitabine (ddC, Hivid, dideoxycytidine), stavudine (d4T, Zerit), lamivudine (3TC, Zeffix, Epivir), tenofovir, abacavir (ABC, Ziagen), emtricitabine (FTC, Emitriva), entecavir (ETV, Baraclude), and apricitabine (ATC).

(II) Non-nucleoside reverse transcriptase inhibitors (NNRTIs). NNRTIs are allosteric inhibitors which bind reversibly at a nonsubstrate-binding site on the reverse transcriptase, thereby altering the shape of the active site or blocking polymerase activity. Examples of NNRTIs include, without limitation, delavirdine (BHAP, U-90152; RESCRIPTOR®), efavirenz (DMP-266, SUSTIVA®), nevirapine (VIRAMUNE®), PNU-142721, capravirine (S-1153, AG-1549), emivirine (+)-calanolide A (NSC-675451) and B, etravirine (TMC-125), rilpivirne (TMC278, Edurant™), delavirdine, DAPY (TMC120), BILR-355 BS, PHI-236, PHI-443 (TMC-278), and lersivirine (UK-453061).

(III) Protease inhibitors (PI). Protease inhibitors are inhibitors of the HIV-1 protease. Examples of protease inhibitors include, without limitation, darunavir, amprenavir (141W94, AGENERASE®), tipranivir (PNU-140690, APTIVUS®), indinavir (MK-639; CRIXIVAN®), saquinavir (INVIRASE®, FORTOVASE®), fosamprenavir (LEXIVA®), lopinavir (ABT-378), ritonavir (ABT-538, NORVIR®), atazanavir (REYATAZ®), nelfinavir (AG-1343, VIRACEPT®), lasinavir (BMS-234475/CGP-61755), BMS-2322623, GW-640385X (VX-385), AG-001859, and SM-309515.

(IV) Viral entry inhibitors. Viral entry inhibitors are compounds which act to block viral entry into the cell. For example, a viral entry inhibitor may be a CCR5 receptor antagonist (e.g., maraviroc (Selzentry®, Celsentri), vicriviroc or CCR5 antibody (e.g., PRO140, HGS004, and HGS101). Viral entry inhibitors also include fusion inhibitors. Fusion inhibitors are compounds, such as peptides, which act by binding to envelope protein (e.g., HIV envelope protein (e.g., gp41, gp120, gp160)) and blocking the structural changes necessary for the virus to fuse with the host cell. Examples of fusion inhibitors include, without limitation, enfuvirtide (INN, FUZEON®), T-20 (DP-178, FUZEON®) and T-1249.

(V) Integrase inhibitors. Integrase inhibitors are a class of antiretroviral drug designed to block the action of integrase, a viral enzyme that inserts the viral genome into the DNA of the host cell. Examples of fusion inhibitors include, without limitation, raltegravir, elvitegravir, S/GSK1265744. S/GSK1349572 (dolutegravir), and MK-2048.

The antiviral may also be a vaccine. For example, the antiretroviral therapeutic may be a vaccine such as an HIV vaccine. HIV vaccines include, without limitation, ALVAC® HIV (vCP1521), AIDSVAX® B/E (120), and combinations thereof. Anti-HIV compounds also include HIV antibodies (e.g., antibodies against gp120 or gp41), particularly broadly neutralizing antibodies.

In a particular embodiment, the anti-HIV agent of the instant invention is an entry inhibitor, protease inhibitor, NNRTI, or NRTI. In a particular embodiment, the anti-HIV agent is selected from the group consisting of maraviroc, indinavir, ritonavir, atazanavir, and efavirenz. As stated hereinabove, more than one antiretroviral therapeutic may be contained with a nanoparticle. When more than one therapeutic agent is used, the agents may have different mechanisms of action or the same mechanism of action (as outlined above). In a particular embodiment, the anti-HIV therapy is highly active antiretroviral therapy (HAART).

As stated hereinabove, the encapsulated compounds can comprise imaging or detection agents, particularly those to be observed or monitored by means other than MRI. For example, the nanoparticles may comprise agents such as radioisotopes, imaging agents, quantum dots, and/or contrast agents. Particular examples include, without limitation: isotopes (e.g., radioisotopes, (e.g., ³H (tritium) and ¹⁴C) or stable isotopes (e.g., ²H (deuterium) ¹¹C, ¹³C, ¹⁷O and ¹⁸O), optical agents, and fluorescence agents. Fluorescent agents include, without limitation, fluorescein and rhodamine and their derivatives. Optical agents include, without limitation, quantum dots, derivatives of phorphyrins, anthraquinones, anthrapyrazoles, perylenequinones, xanthenes, cyanines, acridines, phenoxazines and phenothiazines.

In a particular embodiment of the instant invention, the nanoparticles also comprise a hydrophobic compound (e.g., a hydrophobic polymer or homopolymer) in the core. Hydrophobic compounds can serve as nucleation agents for encapsulated compounds. Examples of hydrophobic polymers include the hydrophobic blocks of the amphiphilic block copolymers set forth hereinbelow. Specific examples of hydrophobic polymers include, without limitation, polyanhydride, polyesters such as polycaprolactone (PCL), poly(lactic acid) (e.g., PDLLA, PLLA, and/or PDLA), and PLGA.

### II. Amphiphilic Compound

As stated hereinabove, the nanoparticles of the instant invention comprise at least one amphiphilic compound or amphiphilic compound plus a hydrophobic compound. The amphiphilic compound may be, for example, a surfactant or a lipid (e.g., a phosholipid), optionally linked to a hydrophilic compound or polymer as described hereinbelow (e.g., PEO, polysaccharide, particularly to the head group). The amphiphilic compound may be charged (positively or negatively) or neutral.

The hydrophobic compound is preferably biocompatible. Examples of biocompatible polymers are known in the art, including, for example, polyanhydride, polyester. Examples of polymers include, without limitation: polyanhydride, polylactic acid (PLA), poly(lactic-co-glycolic acid) (PLGA), polycaprolactone (PCL).

In a particular embodiment, the amphiphilic compound is an amphiphilic copolymer, particularly an amphiphilic block copolymer. Amphiphilic block copolymers may comprise two, three, four, five, or more blocks. For example, the amphiphilic block copolymer may be of the general formula A-B, B-A, A-B-A, B-A-B, A-B-A-B-A, or B-A-B-A-B, wherein A represents a hydrophilic block and B represents a hydrophobic block. The amphiphilic block copolymers may be in a linear formation or a branched, hyper-branched, dendrimer, graft, or star formation (e.g., A(B)n, (AB)n, AnBm starblocks, etc.). In a particular embodiment, the amphiphilic block copolymer comprises hydrophobic blocks at the termini. The blocks of the amphiphilic block copolymers can be of variable length. In a particular embodiment, the blocks of the amphiphilic block copolymer comprise from about 2 to about 800 repeating units, particularly from about 5 to about 200, about 5 to about 150, or about 5 to about 100 repeating units.

The blocks of the amphiphilic block copolymer may comprise a single repeating unit. Alternatively, the blocks may comprise combinations of different hydrophilic or hydrophobic units. Hydrophilic blocks may even comprise hydrophobic units so long as the character of the block is still hydrophilic (and vice versa). For example, to maintain the hydrophilic character of the block, the hydrophilic repeating unit would predominate.

In a particular embodiment, the hydrophilic segments may be polymers with aqueous solubility more that about 1% wt. at 37° C., while hydrophobic segments may be polymers with aqueous solubility less than about 1% wt. at 37° C. In a particular embodiment, polymers that at 1% solution in bi-distilled water have a cloud point above about 37° C., particularly above about 40° C., may be the hydrophilic segments. In a particular embodiment, polymers that at 1% solution in bi-distilled water have a cloud point below about 37° C., particularly below about 34° C., may be the hydrophobic segments.

The amphiphilic compound is preferably biocompatible. Examples of biocompatible amphiphilic copolymers are known in the art, including, for example, those described in Gaucher et al. (J. Control Rel. (2005) 109:169-188). Examples of amphiphilic block copolymers include, without limitation: poly(2-oxazoline) amphiphilic block copolymers, polyethylene glycol-polylactic acid (PEG-PLA), PEG-PLA-PEG, polyethylene glycol-poly(lactic-co-glycolic acid) (PEG-PLGA), polyethylene glycol-polycaprolactone (PEG-PCL), polyethylene glycol-polyaspartate (PEG-PAsp), polyethylene glycol-poly(glutamic acid) (PEG-PGlu), polyethylene glycol-poly(acrylic acid) (PEG-PAA), polyethylene glycol-poly(methacrylic acid) (PEG-PMA), polyethylene glycol-poly(ethyleneimine) (PEG-PEI), polyethylene glycol-poly(L-lysine) (PEG-PLys), polyethylene glycol-poly(2-(N,N-dimethylamino)ethyl methacrylate) (PEG-PDMAEMA), polyethylene glycol-chitosan, and derivatives thereof. Examples of other biocompatible amphiphilic compounds include phospholipids and PEGylated phospholipids.

Examples of hydrophilic block(s) include, without limitation, polyetherglycols, dextran, gelatin, albumin, poly(ethylene oxide), methoxy-poly(ethylene glycol), copolymers of ethylene oxide and propylene oxide, polysaccharides, polyvinyl alcohol, polyvinyl pyrrolidone, polyvinyltriazole, N-oxide of polyvinylpyridine, N-(2-hydroxypropyl)methacrylamide (HPMA), polyortho esters, polyglycerols, polyacrylamide, polyoxazolines (e.g., methyl or ethyl poly(2-oxazolines)), polyacroylmorpholine, and copolymers or derivatives thereof. Examples of hydrophobic block(s) include, without limitation, polyanhydride, polyester, poly(propylene oxide), poly(lactic acid), poly(lactic-co-glycolic acid), poly(lactic-co-glycolide), poly aspartic acid, polyoxazolines (e.g., butyl, propyl, pentyl, nonyl, or phenyl poly(2-oxazolines)), poly glutamic acid, polycaprolactone, poly(propylene oxide), poly(1,2-butylene oxide), poly(n-butylene oxide), poly(ethyleneimine), poly(tetrahydrofurane), ethyl cellulose, polydipyrolle/dicabazole, starch, and/or poly(styrene).

In a particular embodiment, the hydrophilic block(s) of the amphiphilic block copolymer comprises poly(ethylene oxide) (also known as polyethylene glycol) or a polysaccharide. In a particular embodiment, the hydrophobic block(s) of the amphiphilic block copolymer comprises polyanhydride, polyester, poly(lactic acid), polycaprolactone, poly(propylene oxide), poly(1,2-butylene oxide), poly(n-butylene oxide), poly(tetrahydrofurane), and/or poly(styrene).

In a particular embodiment, the amphiphilic block copolymer comprises at least one block of poly(oxyethylene) and at least one block of poly(oxypropylene). In a particular embodiment, the amphiphilic block copolymer is a pentablock copolymer with a middle triblock of poly(oxyethylene)-poly(oxypropylene)-poly(oxyethylene) and terminal hydrophobic blocks.

Polymers comprising at least one block of poly(oxyethylene) and at least one block of poly(oxypropylene) are commercially available under such generic trade names as “lipoloxamers”, “Pluronic®,” “poloxamers,” and “synperonics.” Examples of poloxamers include, without limitation, Pluronic® L31, L35, F38, L42, L43, L44, L61, L62, L63, L64, P65, F68, L72, P75, F77, L81, P84, P85, F87, F88, L92, F98, L101, P103, P104, P105, F108, L121, L122, L123, F127, 10R5, 10R8, 12R3, 17R1, 17R2, 17R4, 17R8, 22R4, 25R1, 25R2, 25R4, 25R5, 25R8, 31R1, 3182, and 31R4. Pluronic® block copolymers are designated by a letter prefix followed by a two or a three digit number. The letter prefixes (L, P, or F) refer to the physical form of each polymer, (liquid, paste, or flakeable solid). The numeric code defines the structural parameters of the block copolymer. The last digit of this code approximates the weight content of EO block in tens of weight percent (for example, 80% weight if the digit is 8, or 10% weight if the digit is 1). The remaining first one or two digits encode the molecular mass of the central PO block. To decipher the code, one should multiply the corresponding number by 300 to obtain the approximate molecular mass in daltons (Da). Therefore Pluronic® nomenclature provides a convenient approach to estimate the characteristics of the block copolymer in the absence of reference literature. For example, the code ‘F127’ defines the block copolymer, which is a solid, has a PO block of 3600 Da (12×300) and 70% weight of EO. The precise molecular characteristics of each Pluronic® block copolymer can be obtained from the manufacturer.

The amphiphilic compound of the instant invention may be linked to at least one targeting ligand. The addition of a targeting ligand and particle size distributions permits improved bioavailability. A targeting ligand is a compound that will specifically bind to a specific type of tissue or cell type. In a particular embodiment, the targeting ligand is a ligand for a cell surface marker/receptor. The targeting ligand may be an antibody or fragment thereof immunologically specific for a cell surface marker (e.g., protein or carbohydrate) preferentially or exclusively expressed on the targeted tissue or cell type. The targeting ligand may be linked directly to the amphiphilic compound or via a linker, particularly to a hydrophilic portion of the amphiphilic compound. Generally, the linker is a chemical moiety comprising a covalent bond or a chain of atoms that covalently attaches the ligand to the amphiphilic compound. The linker can be linked to any synthetically feasible position of the ligand and the amphiphilic compound. Exemplary linkers may comprise at least one optionally substituted; saturated or unsaturated; linear, branched or cyclic alkyl group or an optionally substituted aryl group. The linker may also be a polypeptide (e.g., from about 1 to about 10 amino acids, particularly about 1 to about 5). The linker may be non-degradable and may be a covalent bond or any other chemical structure which cannot be substantially cleaved or cleaved at all under physiological environments or conditions.

Notably, all of the amphiphilic compounds of a nanoparticle need not be linked to a targeting ligand. Indeed, only a portion of the amphiphilic compounds need be linked to a targeting ligand. For example, the ratio of targeting ligand linked to unlinked amphiphilic compounds can be 1:1, 1:2, 1:3, 1:4, 1:5, 1:10, or less. Additionally, the nanoparticles of the instant invention may comprise more than one targeting ligand per nanoparticle. The ratio of the different targeting ligands can be controlled by the ratio of components used to synthesize the nanoparticles (e.g., via flash precipitation).

In a particular embodiment, the targeting ligand is a macrophage targeting ligand. Macrophage targeting ligands include, without limitation, folate receptor ligands (e.g., folate (folic acid) and folate receptor antibodies and fragments thereof (see, e.g., Sudimack et al. (2000) Adv. Drug Del. Rev., 41:147-162)), mannose receptor ligands (e.g., mannose), and formyl peptide receptor (FPR) ligands (e.g., N-formyl-Met-Leu-Phe (fMLF)).

### III. Iron Oxide Particles

The nanoparticles of the instant invention also comprise at least one paramagnetic or superparamagnetic particle or quantum dot. In a particular embodiment, the paramagnetic or superparamagnetic particle comprises iron oxide (e.g., magnetite) or cobalt. In a particular embodiment, the iron oxide particle is a superparamagnetic iron oxide particle (SPIO) or an ultrasmall superparamagnetic iron oxide particle (USPIO). Superparamagnetic iron oxide particles (SPIOs (e.g., ultrasmall superparamagnetic iron oxide particles (USPIOs)) are preferred particles due to their high relaxation values and clinically acceptable biocompatibility (Mahmoudi et al. (2011) Adv. Drug Deliv. Rev., 63:24). SPIOs have been widely used for in vivo biomedical applications including MRI, image-guided drug delivery and hyperthermia therapy (Kievit et al. (2011) Accounts Chem. Res., 44:853; Kumar et al. (2011) Adv. Drug Deliv. Rev., 63:789; Veiseh et al. (2010) Adv. Drug Deliv.

Rev., 62:284). In a particular embodiment, the USPIO has a diameter less than 50 nm, less than about 20 nm, or less than about 10 nm. While iron oxide is exemplified, other metals are paramagnetic and may be used in the instant invention. Examples of paramagnetic metals/ions include, without limitation, gold (e.g., Au(II)), gadolinium (e.g., Gd(III)), europium (e.g., Eu(III)), dysprosium (e.g., Dy(III)), praseodymium (e.g., Pr(III)), protactinium (e.g., Pa(IV)), manganese (e.g., Mn(II)), chromium (e.g., Cr(III)), cobalt (e.g., Co(III)), iron (e.g., Fe(III)), copper (e.g., Cu(II)), nickel (e.g., Ni(II)), titanium (e.g., Ti(III)), and vanadium (e.g., V(IV)).

The small magnetite particles can include oleic acid coated magnetic nanoparticles or other magnetic nanoparticles with hydrophobic coatings (e.g., polymer, lipid, fatty acid, etc.). Indeed, the magnetic particles (e.g., SPIO or USPIO) are preferably hydrophobically modified on the surface (e.g., covalently attached to the surface (e.g., via a linker)). For example, the iron oxide nanoparticles comprise a hydrophobic compound, such as oleic acid, on their surface.

The iron oxide particle of the instant invention may be linked to the encapsulated compound (e.g., therapeutic). The encapsulated compound may be linked directly to the iron oxide particle or its hydrophobic modification or via a linker. Generally, the linker is a chemical moiety comprising a covalent bond or a chain of atoms that covalently attaches the ligand to the surfactant. The linker can be linked to any synthetically feasible position of the iron oxide particle and the encapsulated compound. Exemplary linkers may comprise at least one optionally substituted; saturated or unsaturated; linear, branched or cyclic alkyl group or an optionally substituted aryl group. The linker may also be a polypeptide (e.g., from about 1 to about 10 amino acids, particularly about 1 to about 5). The linker may be non-degradable and may be a covalent bond or any other chemical structure which cannot be substantially cleaved or cleaved at all under physiological environments or conditions.

### IV. Administration

The instant invention encompasses compositions comprising at least one nanoparticle of the instant invention (sometimes referred to herein as SMART) and, optionally, at least one pharmaceutically acceptable carrier. As stated hereinabove, the nanoparticle may comprise more than one encapsulated compound (e.g., therapeutic agent). In a particular embodiment, the composition comprises a first nanoparticle comprising a first encapsulated compound(s) and a second nanoparticle comprising a second encapsulated compound(s), wherein the first and second encapsulated compounds are different. The compositions of the instant invention may further comprise other therapeutic agents (e.g., other antiviral or anti-HIV compounds).

The present invention also encompasses methods for preventing, inhibiting, and/or treating microbial infections (e.g., viral or bacterial (e.g., tuberculosis)), particularly retroviral or lentiviral infections, particularly HIV infections (e.g., HIV-1). The pharmaceutical compositions of the instant invention can be administered to an animal, in particular a mammal, more particularly a human, in order to treat/inhibit a microbial infection. The pharmaceutical compositions of the instant invention may also comprise at least one other anti-microbial agent, particularly at least one other anti-HIV compound/agent. The additional anti-HIV compound may also be administered in separate composition from the anti-HIV nanoparticles of the instant invention. The compositions may be administered at the same time or at different times (e.g., sequentially).

As explained hereinabove, the instant invention also encompasses methods of monitoring pharmacokinetics and biodistribution of the encapsulated compound (e.g., therapeutic agent). In a particular embodiment, the method comprises administering the nanoparticles of the invention to a subject and performing at least one MRI procedure, thereby determining the location of the nanoparticles and the encapsulated compounds. The methods may comprise performing more than one MRI procedure at different times. The methods may further comprise assaying for additional imaging agents, if present. The monitoring of the distribution of the encapsulated compound allows for real time assessment of the therapy (e.g., for personalized medicine) and allow for the optimization of the treatment to direct more of the encapsulated compound to the desired target and reduce toxicity. For example, the route of administration, frequency of administration, amount of dose, and/or targeting of the nanoparticle may be modified.

The dosage ranges for the administration of the compositions of the invention are those large enough to produce the desired effect (e.g., curing, relieving, treating, and/or preventing the HIV infection, the symptoms of it (e.g., AIDS, ARC), or the predisposition towards it). In a particular embodiment, lower doses of the composition of the instant invention are administered, e.g., about 50 mg/kg or less, about 25 mg/kg or less, or about 10 mg/kg or less. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counter indications.

The nanoparticles described herein will generally be administered to a patient as a pharmaceutical preparation. The term “patient” as used herein refers to human or animal subjects. These nanoparticles may be employed therapeutically, under the guidance of a physician. While the therapeutic agents are exemplified herein, any bioactive agent may be administered to a patient, e.g., a diagnostic or imaging agent.

The compositions comprising the nanoparticles of the instant invention may be conveniently formulated for administration with any pharmaceutically acceptable carrier(s). For example, the complexes may be formulated with an acceptable medium such as water, buffered saline, detergents, suspending agents or suitable mixtures thereof. The concentration of the nanoparticles in the chosen medium may be varied and the medium may be chosen based on the desired route of administration of the pharmaceutical preparation. Except insofar as any conventional media or agent is incompatible with the nanoparticles to be administered, its use in the pharmaceutical preparation is contemplated.

The dose and dosage regimen of nanoparticles according to the invention that are suitable for administration to a particular patient may be determined by a physician considering the patient's age, sex, weight, general medical condition, and the specific condition for which the nanoparticles are being administered and the severity thereof. The physician may also take into account the route of administration, the pharmaceutical carrier, and the nanoparticle's biological activity.

Selection of a suitable pharmaceutical preparation will also depend upon the mode of administration chosen. For example, the nanoparticles of the invention may be administered by direct injection or intravenously. In this instance, a pharmaceutical preparation comprises the nanoparticle dispersed in a medium that is compatible with the site of injection.

Nanoparticles of the instant invention may be administered by any method. For example, the nanoparticles of the instant invention can be administered, without limitation parenterally, subcutaneously, orally, topically, pulmonarily, rectally, vaginally, intravenously, intraperitoneally, intrathecally, intracerbrally, epidurally, intramuscularly, intradermally, or intracarotidly. In a particular embodiment, the nanoparticles are administered intravenously or intraperitoneally. Pharmaceutical preparations for injection are known in the art. If injection is selected as a method for administering the nanoparticle, steps must be taken to ensure that sufficient amounts of the molecules or cells reach their target cells to exert a biological effect. Dosage forms for oral administration include, without limitation, tablets (e.g., coated and uncoated, chewable), gelatin capsules (e.g., soft or hard), lozenges, troches, solutions, emulsions, suspensions, syrups, elixirs, powders/granules (e.g., reconstitutable or dispersible) gums, and effervescent tablets. Dosage forms for parenteral administration include, without limitation, solutions, emulsions, suspensions, dispersions and powders/granules for reconstitution. Dosage forms for topical administration include, without limitation, creams, gels, ointments, salves, patches and transdermal delivery systems.

Pharmaceutical compositions containing a nanoparticle of the present invention as the active ingredient in intimate admixture with a pharmaceutically acceptable carrier can be prepared according to conventional pharmaceutical compounding techniques. The carrier may take a wide variety of forms depending on the form of preparation desired for administration, e.g., intravenous, oral, direct injection, intracranial, and intravitreal.

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art.

Dosage units may be proportionately increased or decreased based on the weight of the patient. Appropriate concentrations for alleviation of a particular pathological condition may be determined by dosage concentration curve calculations, as known in the art.

In accordance with the present invention, the appropriate dosage unit for the administration of nanoparticles may be determined by evaluating the toxicity of the molecules or cells in animal models. Various concentrations of nanoparticles in pharmaceutical preparations may be administered to mice, and the minimal and maximal dosages may be determined based on the beneficial results and side effects observed as a result of the treatment. Appropriate dosage unit may also be determined by assessing the efficacy of the nanoparticle treatment in combination with other standard drugs. The dosage units of nanoparticle may be determined individually or in combination with each treatment according to the effect detected.

The pharmaceutical preparation comprising the nanoparticles may be administered at appropriate intervals, for example, at least twice a day or more until the pathological symptoms are reduced or alleviated, after which the dosage may be reduced to a maintenance level. The appropriate interval in a particular case would normally depend on the condition of the patient.

The instant invention encompasses methods of treating a disease/disorder comprising administering to a subject in need thereof a composition comprising a nanoparticle of the instant invention and, particularly, at least one pharmaceutically acceptable carrier. Nanoparticles of the instant invention can be injected directly to a subject or through injection with macrophages that have internalized nanoparticles ex vivo/in vitro. In a particular embodiment of the instant invention, the instant methods comprise treating the subject via an ex vivo therapy. In particular, the method comprises removing cells from the subject, exposing/contacting the cells in vitro to the nanoparticles of the instant invention, and returning the cells to the subject. In a particular embodiment, the cells comprise macrophage. Other methods of treating the disease or disorder may be combined with the methods of the instant invention may be co-administered with the compositions of the instant invention.

The instant also encompasses delivering the nanoparticle of the instant invention to a cell in vitro (e.g., in culture). The nanoparticle may be delivered to the cell in at least one carrier.

### V. Definition

The singular forms “a,” “an,” and “the” include plural referents unless the context clearly dictates otherwise.

As used herein, the term “subject” refers to an animal, particularly a mammal, particularly a human.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., Tween 80, Polysorbate 80), emulsifier, buffer (e.g., Tris HCl, acetate, phosphate), antimicrobial, bulking substance (e.g., lactose, mannitol), excipient, auxiliary agent or vehicle with which an active agent of the present invention is administered. Pharmaceutically acceptable carriers can be sterile liquids, such as water and oils, including those of petroleum, animal, vegetable or synthetic origin. Water or aqueous saline solutions and aqueous dextrose and glycerol solutions may be employed as carriers, particularly for injectable solutions. Suitable pharmaceutical carriers are described in “Remington's Pharmaceutical Sciences” by E. W. Martin (Mack Publishing Co., Easton, Pa.); Gennaro, A. R., Remington: The Science and Practice of Pharmacy, (Lippincott, Williams and Wilkins); Liberman, et al., Eds., Pharmaceutical Dosage Forms, Marcel Decker, New York, N.Y.; and Kibbe, et al., Eds., Handbook of Pharmaceutical Excipients, American Pharmaceutical Association, Washington.

The term “treat” as used herein refers to any type of treatment that imparts a benefit to a patient afflicted with a disease, including improvement in the condition of the patient (e.g., in one or more symptoms), delay in the progression of the condition, etc. In a particular embodiment, the treatment of a retroviral infection results in at least an inhibition/reduction in the number of infected cells.

As used herein, the term “prevent” refers to the prophylactic treatment of a subject who is at risk of developing a condition (e.g., microbial pathogen infection) resulting in a decrease in the probability that the subject will develop the condition.

A “therapeutically effective amount” of a compound or a pharmaceutical composition refers to an amount effective to prevent, inhibit, treat, or lessen the symptoms of a particular disorder or disease. The treatment of a microbial infection (e.g., HIV infection) herein may refer to curing, relieving, and/or preventing the microbial infection, the symptom(s) of it, or the predisposition towards it.

As used herein, the term “therapeutic agent” refers to a chemical compound or biological molecule including, without limitation, nucleic acids, peptides, proteins, and antibodies that can be used to treat a condition, disease, or disorder or reduce the symptoms of the condition, disease, or disorder.

As used herein, the term “small molecule” refers to a substance or compound that has a relatively low molecular weight (e.g., less than 4,000, less than 2,000, particularly less than 1 kDa or 800 Da). Typically, small molecules are organic, but are not proteins, polypeptides, or nucleic acids, though they may be amino acids or dipeptides.

The term “antimicrobials” as used herein indicates a substance that kills or inhibits the growth of microorganisms such as bacteria, fungi, viruses, or protozoans.

As used herein the term “antibiotic” refers to a molecule that inhibits bacterial growth or pathogenesis. Antibiotics include, without limitation, β-lactams (e.g., penicillins and cephalosporins), vancomycins, bacitracins, macrolides (e.g., erythromycins, clarithromycin, azithromycin), lincosamides (e.g., clindomycin), chloramphenicols, tetracyclines (e.g., immunocycline, chlortetracycline, oxytetracycline, demeclocycline, methacycline, doxycycline and minocycline), aminoglycosides (e.g., gentamicins, amikacins, neomycins, amikacin, streptomycin, kanamycin), amphotericins, cefazolins, clindamycins, mupirocins, sulfonamides and trimethoprim, rifampicins, metronidazoles, quinolones, fluoroquinolones (e.g., ciprofloxacin, levofloxacin, moxifloxacin), novobiocins, polymixins, gramicidins, vancomycin, imipenem, meropenem, cefoperazone, cefepime, penicillin, nafcillin, linezolid, aztreonam, piperacillin, tazobactam, ampicillin, sulbactam, clindamycin, metronidazole, levofloxacin, a carbapenem, linezolid, rifamycins (e.g., rifampin, rifabutin), clofazimine, and metronidazole.

As used herein, the term “antiviral” refers to a substance that destroys a virus or suppresses replication (reproduction) of the virus.

As used herein, the term “highly active antiretroviral therapy” (HAART) refers to HIV therapy with various combinations of therapeutics such as nucleoside reverse transcriptase inhibitors, non-nucleoside reverse transcriptase inhibitors, HIV protease inhibitors, and fusion inhibitors.

As used herein, the term “amphiphilic” means the ability to dissolve in both water and lipids/polar environments. Typically, an amphiphilic compound comprises a hydrophilic portion and a hydrophobic portion. “Hydrophobic” designates a preference for apolar environments (e.g., a hydrophobic substance or moiety is more readily dissolved in or wetted by non-polar solvents, such as hydrocarbons, than by water). As used herein, the term “hydrophilic” means the ability to dissolve in water.

As used herein, the term “polymer” denotes molecules formed from the chemical union of two or more repeating units or monomers. The term “block copolymer” most simply refers to conjugates of at least two different polymer segments, wherein each polymer segment comprises two or more adjacent units of the same kind.

An “antibody” or “antibody molecule” is any immunoglobulin, including antibodies and fragments thereof (e.g., scFv), that binds to a specific antigen. As used herein, antibody or antibody molecule contemplates intact immunoglobulin molecules, immunologically active portions of an immunoglobulin molecule, and fusions of immunologically active portions of an immunoglobulin molecule.

As used herein, the term “immunologically specific” refers to proteins/polypeptides, particularly antibodies, that bind to one or more epitopes of a protein or compound of interest, but which do not substantially recognize and bind other molecules in a sample containing a mixed population of antigenic biological molecules.

The following examples provide illustrative methods of practicing the instant invention, and are not intended to limit the scope of the invention in any way.

### EXAMPLE 1

### Materials and Methods

### Material Preparation and Characterization

PLGA, 1,2-distearoyl-sn-glycero-3-phospho-choline (DSPC) and 1,2-distearoyl-sn-glycero-3-phosphoethanolamine-N-[methoxy-(polyethylene glycol)-2000] (DSPE-PEG₂₀₀₀) encased the SMART particle containing ATV and magnetite. The magnetite particles were synthesized as follows: 6 mmol tris(acetylacetonato) iron(III), abbreviated as Fe(acac)3 was mixed with 30 mmol 1,2-hexadecanediol, 18 mmol oleic acid, 18 mmol olylamine and 60 mL benzyl ether in a three-neck round-bottomed flask equipped with condenser, magnetic stirrer, thermograph, heating mantle and stirred under nitrogen. The mixture was slowly heated to 110° C. and kept at that temperature for 1 hour, then slowly heated to 200° C. Reflux was kept after it reached 200° C. for 2 hours, then slowly heated to 298° C. and kept at reflux for another 1.5 hours. After cooling down to room temperature, a dark homogeneous colloidal suspension was obtained. The suspension was precipitated in ethanol with a magnetic field. The black precipitate was dissolved in hexane with the presence of oleic acid and oleylamine and the solution was centrifuged at 3,800×g for 10 minutes to remove any undispersed residue. The black solution was re-precipitated in ethanol and centrifuged at 10,000×g for 30 minutes. Solid products were obtained by drying the precipitate under vacuum, generating the final dry particles. Image analysis of ˜200 particles from TEM micrographs indicate that the mean diameter is 8.99±0.32 nm.

### SMART Composition and Characterization

Preparation of the drug loaded DSPC/mPEG-DSPE shell and PLGA core particle was as follows. First, a weighed amount of PLGA, ATV and magnetite were dissolved in chloroform (oil phase) with a weight ratio of magnetite to ATV of 1:3. Second, the aqueous phase was prepared by hydration of DSPC and mPEG-DSPE films. The oil phase was added to the DSPC and mPEG-DSPE aqueous solution drop-by-drop with constant stirring then sonicated for 60 seconds followed by a 20 second break under an ice bath. This procedure was repeated for three cycles. Chloroform was then removed by stirring overnight. Third, the particle suspension was centrifuged at 500×g for 5 minutes. The supernatant fluids were collected to remove the aggregated nanoparticles. A high speed 50,000×g centrifugation for 20 minutes was used to collect the nanoparticles. After washing twice with phosphate-buffered saline (PBS), the nanoparticles were resuspended. SMART size and size distribution were measured by dynamic light scattering (DLS, 90Plus, Brookhaven Instruments Co. USA) then diluted in ultrapure water related to mass concentrations and dispersions. Fourth, the surface charge of the SMART particles, was determined by ZetaPlus, a zeta-potential analyzer (Brookhaven Instruments Co. USA). The pH value and concentration of the particles dispersion were fixed before measurements of zeta potentials. Fifth, the shape and surface morphology of the SMART particles were investigated by transmission electron microscopy (Nowacek et al. (2011) J. Control Release 150:204-211). Samples were prepared from dilutions in distilled water of particle suspensions and dropped onto stubs. After air drying the particles were coated with a thin layer of gold then examined by transmission electron microscopy. The magnetic properties were determined by a Physical Property Measurement System (Boska et al. (2010) J. Vis. Exp., 9(46):2459).

Drug Stability and Release in Isotonic Solution from SMART Particles

SMART particles were dispersed in phosphate buffered saline (PBS, pH 7.4). The dispersion was placed into a 10 k dialysis tube in PBS under stirring at 37° C. At 30 minutes, 1, 2, 3, 4, 6, 8 and 10 days, 100 μl of the suspension was collected. The supernatant was dissolved in THF/methanol (volume ratio 1:10) mixture. The amount of ATV and magnetite was measured by high performance liquid chromatography (HPLC) and inductively coupled plasma mass spectrometry (ICP-MS), respectively (Mascheri et al. (2009) Magn. Reson. Imaging 27:961-969; Nowacek et al. (2011) J. Control Release 150:204-211).

### SMART Uptake and Retention by MDM

Human monocytes were obtained by leukapheresis, from HIV-1 and hepatitis B sero-negative donors, then purified by counter-current centrifugal elutriation (Beduneau et al. (2009) PLoS One 4:e4343). Monocytes were cultured in 6-well plates at a density of 1×10⁶ cells/ml in DMEM containing 10% heat-inactivated pooled human serum, 1% glutamine, 50 μg/ml gentamicin, 10 μg/ml ciprofloxacin and 1,000 U/ml recombinant human macrophage-colony stimulating factor (Gendelman et al. (1988) J. Exp. Med., 167:1428-1441). After 7 days of differentiation, MDM were treated with 100 μM SMART particles, (based upon ATV content). Uptake of SMART particles was assessed without medium change for 8 hours. Adherent MDM were collected by scraping into PBS, at 1, 2, 4 and 8 hours after treatments. Cells were pelleted by centrifugation at 1000×g or 8 minutes at 4° C. Cell pellets were briefly sonicated in 200 μl of methanol/acetonitrile (1:1) and centrifuged at 16,000 rpm for 10 minutes at 4° C. To determine cell retention of SMART particles, MDM were exposed to 100 μM SMART particles for 8 hours, washed 3× with PBS, and fresh media without particles was added. MDM were cultured for an additional 15 days with half medium exchanges every other day. On days 1, 5, 10 and 15 after SMART treatment, MDM were collected as described for cell uptake. Cell extracts were stored at −80° C. until HPLC analysis (Nowacek et al. (2011) J. Control Release 150:204-211).

### Prussian Blue Staining of MDM Retained SMART Particles

MDM were treated with 100 μM SMART particles for 24 hours. Adherent MDM were washed 3× with PBS. Cells were fixed with 2% formalin/2.5% glutaraldehyde in PBS for 10 minutes then washed 2× with PBS. Stained fixed macrophages were treated with 5% potassium ferrocyanide/5% hydrochloric acid (1:1) for 10 minutes at room temperature. Following solution aspiration the cells were washed 2× with PBS. Stained cells were examined by light microscopy.

### MRI Phantoms and Relaxivity Measures

MDM were seeded onto 12-well plates at 1×10⁶ cells/ml. After the cells reached 80% confluence, the medium was changed to medium containing 100 μM SMART particles (based on ATV content). Twenty-four hours later the treatment medium was removed and the cells were washed 3× with 1 ml PBS. Cells were collected and suspended at different cell concentrations (0-5×10⁶ cells/ml) in 1% agar gel. T₂-relaxivity was measured by MRI. Magnetite content in the cells was quantitated by ICP-MS.

### SMART Biodistribution

Biodistribution of SMART particles was determined in male Balb/cJ mice (Jackson Labs, Bar Harbor, Me.). SMART particles (30 mg/kg ATV) were injected via a jugular vein cannula in a total volume of 100 μl for each mouse. The mice were scanned by MRI two hours before injection then continuously at 0.25, 1, 2 and 4 hours or at 24 hours after SMART administration. Tissues were collected following the final MRI scan. Tissue drug levels were quantitated by ultra performance liquid chromatography tandem mass spectrometry (UPLC-MS/MS) (Huang et al. (2011) J. Chromatogr. B Analyt. Technol. Biomed. Life Sci., 879:2332-2338) and magnetite levels were determined by ICP-MS (Mascheri et al. (2009) Magn. Reson. Imaging 27:961-969).

### MRI Acquisition

MRI was acquired using a 7T/16cm Bruker (Ettlingen, Germany) Pharmascan MRI/MRS scanner and a commercial mouse body resonator. SMART detection by MRI was done using T₂ mapping for quantitation and T₂* weighted high resolution imaging for detection of biodistribution throughout the body. The sequence used for T₂ mapping was a CPMG phase cycled multislice multiecho sequence. Forty-one 0.5 mm thick contiguous interleaved coronal images were acquired with an acquisition matrix of 256×192, 40 mm field of view, 12 echoes at 10 ms first echo time and 10 ms echo spacing, repetition time of 5500 ms, one average, for a total acquisition time of 17 ms. T₂* weighted MRI was acquired using a 3D spoiled gradient recalled echo sequence with echo time=3 ms, repetition time, 10 ms repetition time, 15 degree pulse angle, 50×40×30 mm FOV, 256×196×128 acquisition matrix, six averages, for a total scan time of 25 minutes.

### MRI Analyses

T₂ maps were reconstructed using custom programs written in Interactive Data Language (IDL; Exelis Visual Information Solutions, McLean, Va.). Preinjection and 24 hour postinjection maps were constructed using the even-echo images from the CPMG phase cycled imaging data set. Mean tissue T₂ was determined using region of interest analyses before and after SMART injection for the 24 hour results. Magnetite concentration was then determined from the change in relaxivity (ΔR₂=1/T_{2preinjection}−1/T_{2postinjection}) and the per milligram iron of SMART particle relaxivity (r₂) determined as the slope of magnetite concentration versus R₂ in phantom studies. Acute (0-4 hour) data were acquired with in-magnet jugular vein injection, allowing sequential T₂ mapping to be acquired with a T₂* weighted FLASH image acquired at the end of a four-hour period. The natural coregistration of these data allowed development of magnetite concentration maps based on relaxivity changes using custom programs written in IDL for the acute scanning session. The ROI analyses were performed using Image) (imagej.nih.gov/lj) software. For analysis of the acute study, the windows synchronize option was used to simultaneously draw ROIs at same locations on all concentration maps at different time points.

### Immunohistochemical Identification of Cell-SMART Uptake

To determine cell localization of SMART spleen and liver were collected after the final MRI scan and fixed in 10% neutral buffered formalin. Tissues were paraffin embedded and sectioned at 5 μm. To identify macrophages, sections were incubated with ionized calcium binding adaptor molecule 1 (Iba1, Wako Chemicals USA, Inc., Richmond, Va.) for brightfield imaging. The polymer-based HRP-conjugated anti-mouse and anti-rabbit Dako EnVision™ were used as secondary detection reagents and color developed with 3,3′-diaminobenzidine (DAB). All paraffin-embedded sections were stained with Prussian blue to identify magnetite content. Slides were imaged using a Nuance light microscopy system.

### Results

A schematic structure of SMART is represented in FIG. 1A. This is composed of a hydrophobic PLGA/ATV/magnetite core and an amphiphilic DSPC and DSPE-PEG2 k lipid shell. DSPC and DSPE-PEG2 k increased SMART stability and facilitated increased systemic formulation circulation times. Both ATV and magnetite are distributed homogeneously within the core of the particle. SMART was made using a single oil-in-water emulsion with lipid surfactants. After sonication amphiphilic lipids self-assembled to the monolayer surrounding PLGA/ATV/magnetite containing oil droplets, achieved through hydrophobic interactions. Evaporation of chloroform under continuous magnetic stirring allowed for the formation of lipid-coated solid PLGA/ATV/magnetite core. SMART was then purified by ultracentrifugation before further characterization. The DLS results showed that the average size of the particles is 268 nm with a polydispersity of 0.2. The narrow size distribution is linked to the DSPC, which serves to stabilize the polymeric SMART in the aqueous phase. The zeta potential of the particles is −45.2 mV, which provides its stability when suspended in aqueous media. Although DSPC is neutral when it is used as a particle coat it exhibits non-zero mobilities in an external electric field. This may result in a higher negative charge since some anions bind to the neutral lipids making the surface more negatively charged. Transmission electron microscopy (TEM) was employed to obtain the image that best reflects SMART particle morphology (FIG. 1B, right panel). This illustrated that the particles were spherical in shape with narrow size distributions. A representative particle is shown by TEM and showing the ultra small iron oxide contained within the particle's core.

The preliminary in vitro results showed that SMART is very stable and ATV can slowly release from SMART up to 10 days. After SMART particle characterizations were completed, the in vitro kinetics of MDM uptake and retention were determined Studies of nanoART uptake in MDM showed that >95% of total uptake occurs by 8 hours for ATV nanoART (Nowacek et al. (2011) J. Control Release 150:204-211; Balkundi et al. (2011) Int. J. Nanomed., 6:3393-3404; Nowacek et al. (2010) J. Neuroimmune Pharmacol., 5:592-601; Nowacek et al. (2009) Nanomed., 4:903-917). Up to 2 μg of ATV/10⁶ cells was recorded in MDM at 8 hours with magnetite uptake reflective of particle composition (FIG. 1C). The majority of the MDM took up the magnetite as observed through Prussian blue staining (FIG. 1D). Indeed, such staining demonstrated that magnetite containing particles were readily incorporated in macrophages by 8 hours. The controlled and sustained release profile or ATV facilitates the application of the SMART particles for the delivery of antiretroviral drugs.

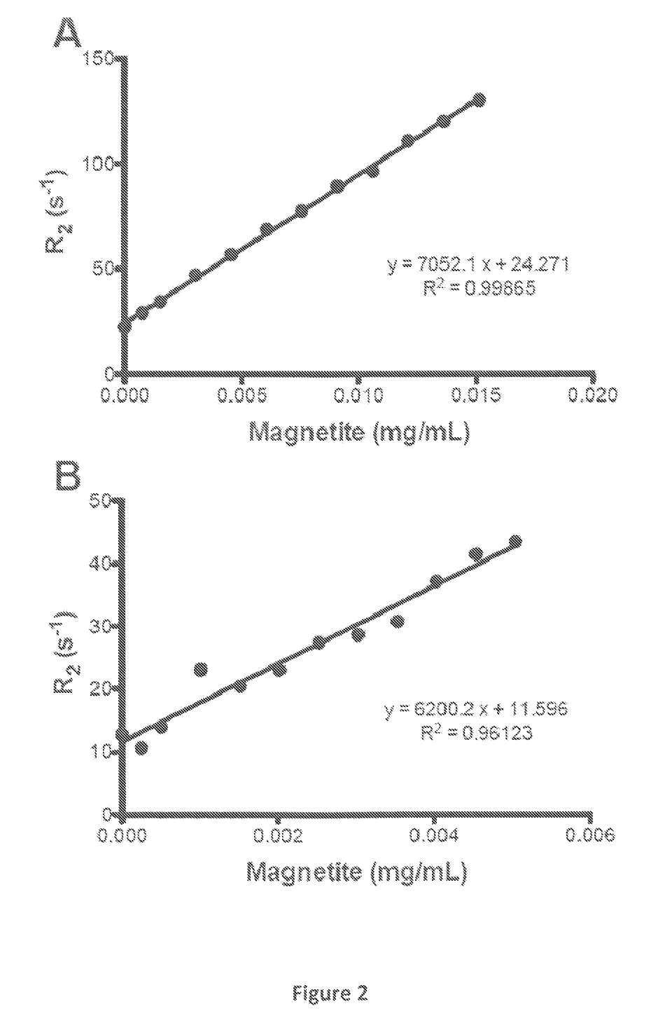

Concentration dependant relaxivity (r₂ (s⁻¹ ml mg⁻¹)) causing increased relaxivity (R₂ (s⁻¹)) in tissue as a function of concentration (expressed as mg/ml magnetite) of SMART particles were determined using phantoms consisting of both free SMART particles and SMART particles taken up by MDM (FIG. 2). The magnetite concentrations in mg/ml of SMART in 1% agar gels were plotted against R₂ as measured by MRI. The relationship between R₂ and magnetite concentration of SMART in phantoms was linear within the range of the measured magnetite concentrations. The concentration dependant relaxivity of SMART was found to be r₂=5993.2 (s⁻¹ ml mg⁻¹) in MDM and r₂=6816.6 (s⁻¹ ml mg⁻¹) in PBS. The r₂ of SMART enables noninvasive in-vivo quantitation of magnetite concentration due to SMART influx using MRI.

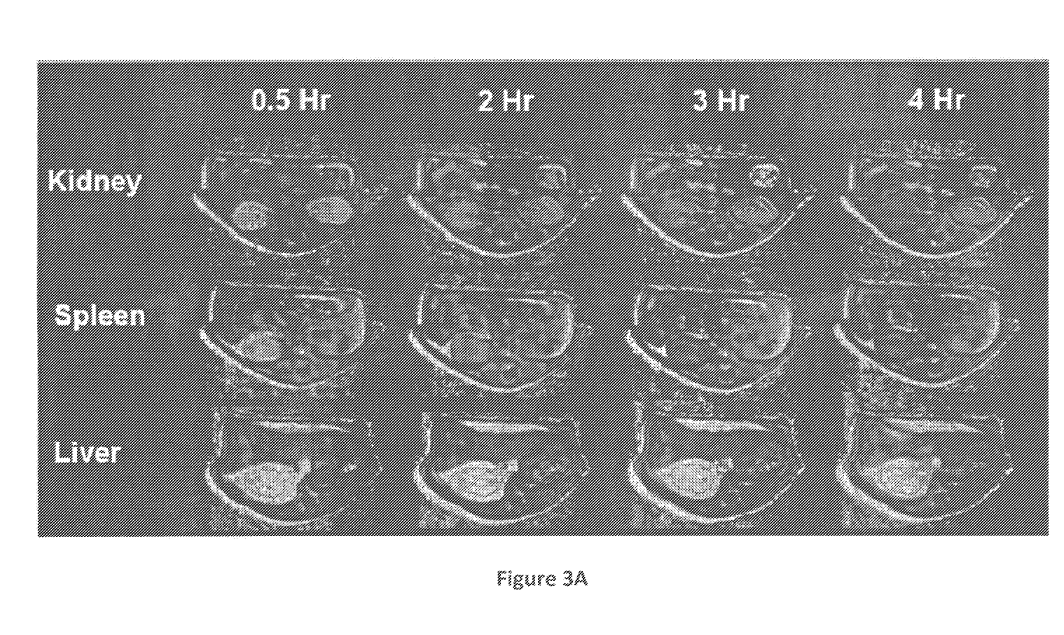

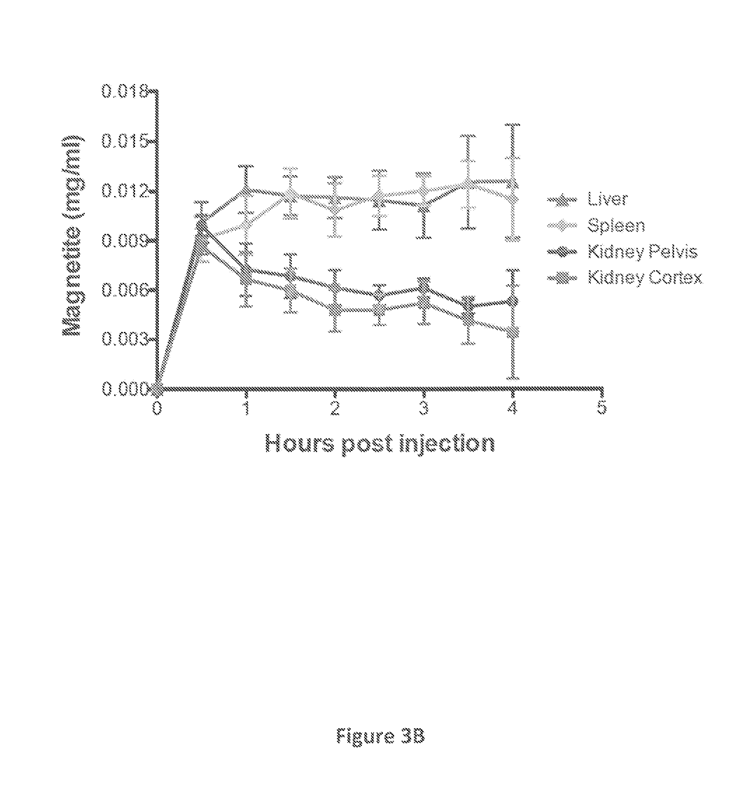

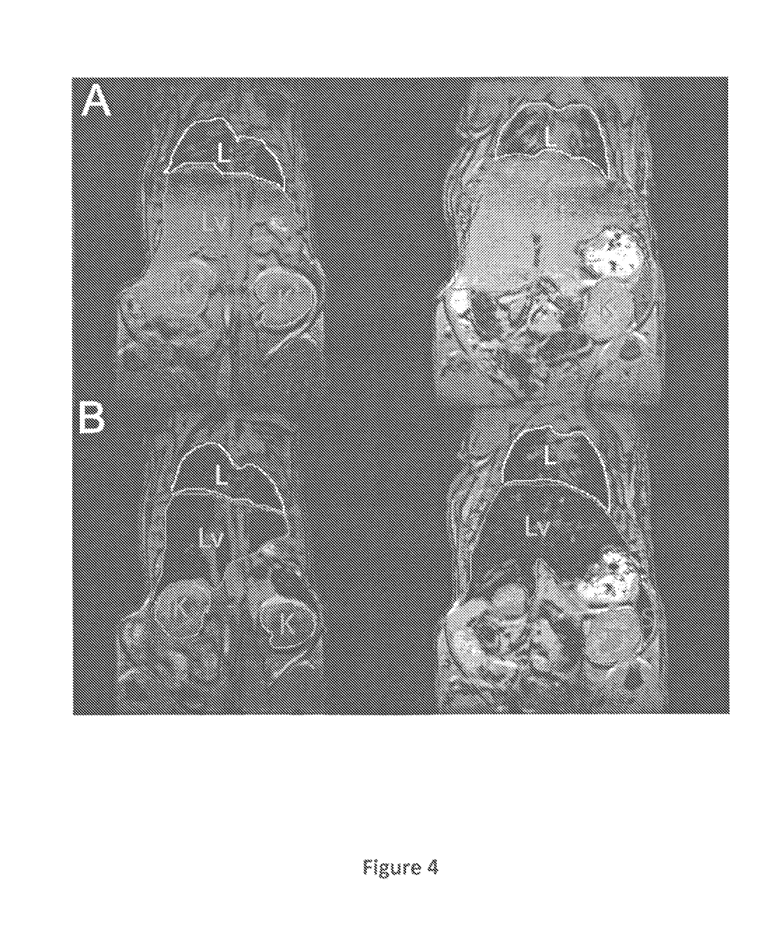

Magnetite labeling allows MRI to be used to quantify the distribution of SMART particles over time in live animals. This can be seen in FIG. 3. FIG. 3A shows examples of magnetite concentration (from magnetite in SMART) constructed from MRI T₂ maps measured before and continuously every 30 minutes for four hours after SMART injection. Region of interest analyses of these data from six animals are shown in FIG. 3B. It can be appreciated from the images that a significant amount of the SMART is still within the vasculature, largely leading to the intensity in the kidney, as kidney shows very little uptake by 24 hours. This reflects the measured concentration in kidney reducing over the first four hours while in liver and spleen, organs where SMART accumulates, the mean signal is relatively constant or increases as the particles redistribute from the blood to the tissue. Significant accumulation of SMART was found in liver and spleen at 4 hours as can be appreciated in FIG. 4. FIG. 4 displays T₂* weighted high resolution 3D FLASH images of the same mouse before and 4 hours after injection of SMART. Presence of magnetite in tissue causes a reduction of T₂* to the point of complete signal loss at TE=3 ms in the liver, spleen, and some abdominal regions. This method is not quantitative, however it does allow ready identification of the presence of magnetite through the body which can be used to guide quantitative region of interest analyses from T₂ maps.

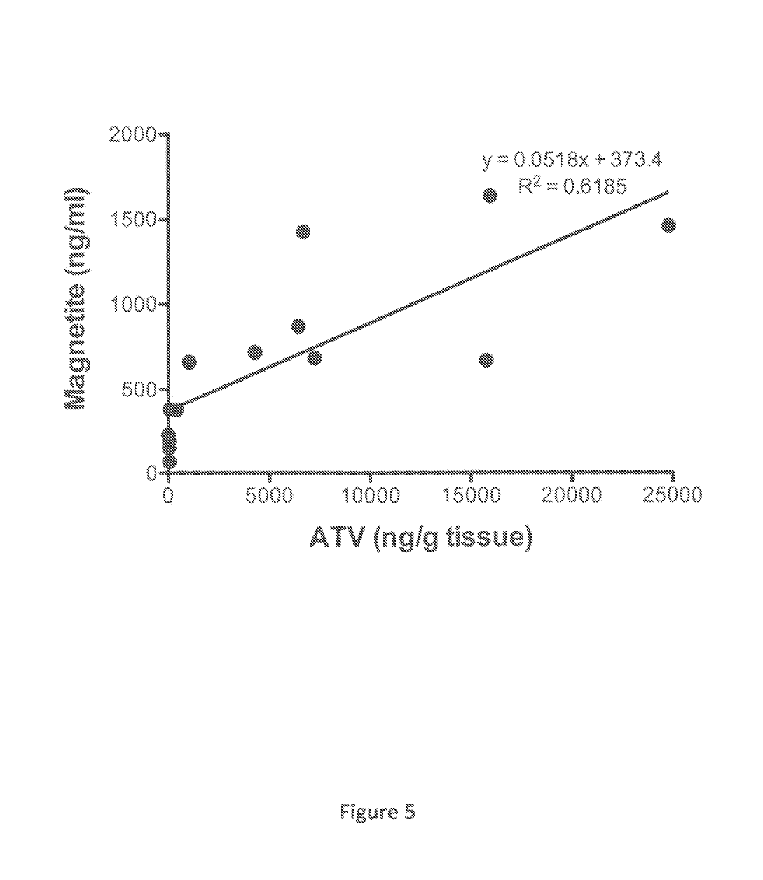

FIG. 5 shows the relationship between magnetite concentration and ATZ concentration of liver, spleen and kidney in four animals 24 hours after injection. It can be appreciated that there is a significant positive correlation (Pearson Correlation, r=0.789, p=0.0013). These results demonstrate the capability of MRI to be used for monitoring nanoART distribution.

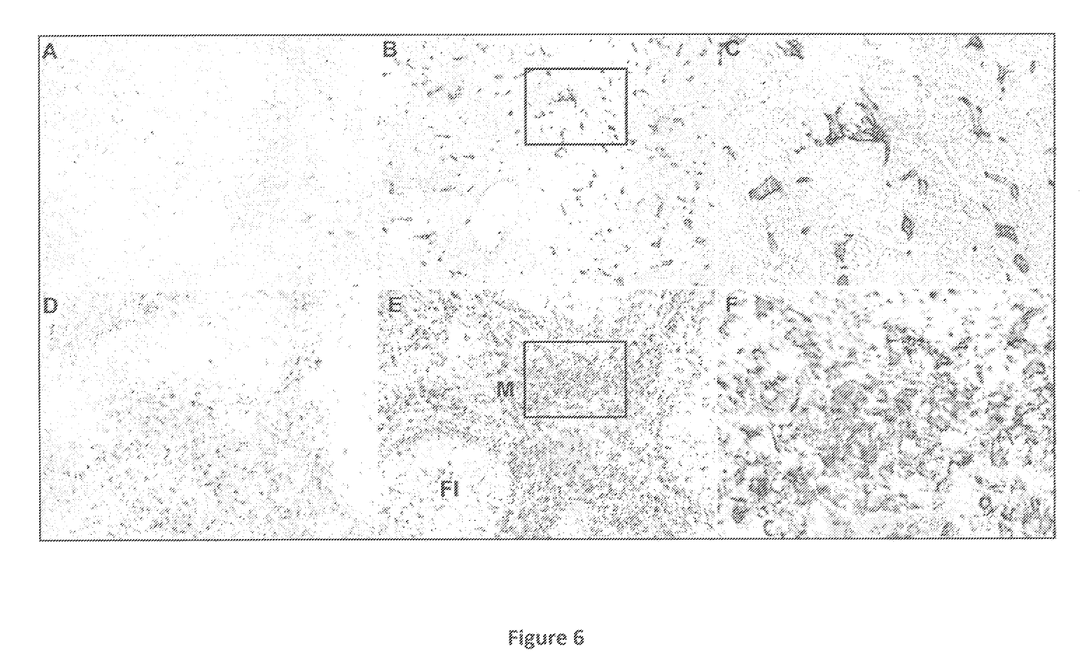

Cellular biodistribution of SMART was concordant with the results observed with nanoART (Roy et al. (2012) J. Infect. Dis., 206:1577-1588). To further show this, the relationships between SMART particle biodistribution and macrophages in mice following parenteral SMART injections were studied. Animals were sacrificed 4 hours after injection and tissues collected. Dual Iba-1 (for macrophages) and Prussian blue staining (for magnetite) were performed and evaluated by bright field microscopic imaging. Prussian blue staining was nearly exclusively in tissue cells identified as macrophages. As shown in FIG. 6, Iba-1⁺ macrophages were readily seen in both liver and spleen in replicate distributions of Prussian blue. The dual staining pictures showed that the SMART particles were retained in tissue macrophages.

Cell-based carriage and delivery of antiretroviral drugs to sites of active HIV-1 replication has been described (Nowacek et al. (2011) J. Control Rel.,150:204-211; Beduneau et al. (2009) PLoS One 4:e4343; Balkundi et al. (2011) Int. J. Nanomed., 6:3393-3404; Nowacek et al. (2009) Nanomed., 4:903-917; Dash et al. (2012) AIDS 26:2135-2144; Dou et al. (2009) J. Immunol., 183:661-669; Roy et al. (2012) J. Infect. Dis., 206:1577-1588). This so-called “Trojan Horse Macrophage” drug delivery scheme takes full advantage of the cells' substantive endosomal storage capacity, its phagocytic and secretory functions, and its high degree of mobility to facilitate drug delivery (Kadiu et al. (2011) Nanomed., 6:975-994). As the macrophage is a principal cell target for viral growth, the added benefit rests in the abilities to bring ART to subcellular sites of viral assembly (Gendelman et al. (2003) The neurological manifestations of HIV-1 infection, Lippincott-Raven Publishers, Philadelphia, 2003). Such a system when used as a weekly or monthly parenteral injection has previously been shown to hold significant gains over conventional native oral drug therapeutic regimens (Dash et al. (2012) AIDS 26:2135-2144; Roy et al. (2012) J. Infect. Dis., 206:1577-1588).

The instant system allows for the utilization of MRI tests to rapidly assess cell and tissue drug biodistribution. The polymer-encased dual magnetite and drug particle permits a clear determination of drug levels in virus-target tissues in a very short time interval (hours). As plasma drug levels remain the gold standard for pharmacokinetic testing this technology clearly opens new opportunities to develop platforms that would accelerate elimination or cure of viral infections. Notably, there is a considerable focus amongst HIV/AIDS researchers towards the development of any or all reliable methods to bring drugs to reservoir sites with the explicit goal of eliminating virus. Targeted drug as well as gene delivery when combined with suitable imaging techniques could facilitate this goal by providing an immediate assessment for treatment success (Nowacek et al. (2011) J. Control Release 150:204-211). Although this is the first time such “theranostics” has been applied for HIV diagnosis and therapies, other systems have been developed in recent years for cancer treatments (Choi et al. (2012) Nanoscale 4:330-342). Here, the application is for early diagnostics. The unique properties of nanomaterials include fluorescent semiconductor nanocrystals (quantum dots) as well as the kind of magnetic nanoparticles developed in this report. All provide properties that can facilitate in vivo imaging with the help of MRI tests as well as fluorescence based approaches. In all, the instant invention allows for the development of carrier particles designed to target specific tissue and effect local chemo-, radio- and gene-directed antiretroviral or immune modulatory therapies.

Liposomes and polymer nanoparticles are the two major types of drug delivery systems (DDS) that have been developed and evaluated for diagnostic and therapeutic purposes. Liposomes composed of natural lipids are attractive DDS because of their high biocompatibility, low immunogenicity, long systemic circulation, favorable pharmacokinetic profile. Specific targeted delivery can be easily achieved by conjugating a targeting ligand to the lipid molecule (Barenholz et al. (2012) J. Controlled Rel., 160:117-134; Lasic, D. D. (1996) Nature 380:561-562; Torchilin, V. P. (2005) Nature Rev., 4:145-160). Several liposomal drug formulations have been approved by FDA for clinical application, such as Doxil and DaunoXome (Barenholz et al. (2012) J. Controlled Rel., 160:117-134; Torchilin, V. P. (2005) Nature Rev., 4:145-160; Petre, D. P. (2007) Intl. J. Nanomed., 2:277-288). However, the possible intrinsic low drug loading capacity, fast release profiles of hydrophobic drugs and physical instability of liposomes limit their clinical applications of different drugs (Liu et al. (2010) Intl. J. Pharm., 395:243-250). Polymeric nanoparticles composed of synthetic PLGA are another widely developed/studied drug delivery platform because of their high stability, relatively high drug loading capacity of all kinds of drugs, biodegradability, low toxicity, and controlled/sustained drug release profiles. Depending on particle composition, the drug release profiles of PLGA nanoparticles can be modulated within days, weeks or even months (Avgoustakis (2004) Current Drug Del., 1:321-333; Cho et al. (2008) Clin. Cancer Res., 14:1310-1316; Panyam et al. (2003) Adv. Drug Del. Rev., 55:329-347). However, the biocompatibility/immunogenicity of nanoparticles composed of synthetic polymers including PLGA is not as high as liposomes. Without further chemical modification, PLGA nanoparticles are rapidly removed from circulation by the mononuclear phagocyte system (MPS), resulting in short systemic circulation (Liu et al. (2010) Intl. J. Pharm., 395:243-250). Generally speaking, both liposomes and PLGA nanoparticles are not independently structurally robust platforms. Thus, lipid-coated polymer nanoparticles, formed by combining synthetic polymers and natural lipids, have been developed as robust drug delivery platform to combine the advantages and avoid the disadvantages of liposomes and polymer nanoparticles (Chan et al. (2009) Biomaterials 30:1627-1634; Li et al. (2012) Intl. J. Nanomed., 7:187-197).

The visualization of cellular function in living organisms has been performed (Beduneau et al. (2009) PLoS One 4:e4343; Kingsley et al. (2006) J. Neuroimmune Pharmacol., 1:340-350; Wessels, J. C. (2007) Semin. Cell Dev. Biol., 18:412-423). Optical, X-ray, nuclear, MRI and ultrasound allows three-dimensional whole-body scans at high spatial resolution and is adept at morphological and functional evaluations. The data obtained can be enhanced by magnetite and image resolution. By immobilizing a specific target molecule on the surface of a magnetic particle, the molecule inherits its magnetic property. Magnetic tissue targeting using multifunctional carrier particles can also facilitate effective treatments by enabling site-directed therapeutic outcomes. To this end, DSPC and DSPE-PEG2 k were selected as the shell and PLGA as the core of SMART system. DSPC is used to increase the biocompatibility of SMART, and DSPE-PEG2 k is used to build a sterically repulsive shield in SMART that make SMART has the ability to reduce opsonization, prevent interactions with the MPS, escape renal exclusion, and increase systemic circulation. This is the first use of lipid-coated PLGA nanoparticles in the HIV field. Lipid coated PLGA to encase magnetite and antiretroviral therapy to facilitate MDM uptake of drug and its subsequent slow release. The synthesized SMART may be used to facilitate drug screening for specific targeting ligands or sugars. SMART may also be used to determine the distribution of nanoART in viral reservoirs for the ultimate eradication of HIV.

### EXAMPLE 2

SMART nanoparticles were fabricated with a rapid precipitation process and also a slow dialysis method. The rapid process allows for narrow particle size distributions. Thus, nanoparticles containing magnetite and/or ritonavir (RTV) were prepared by “flash nanoprecipitation” with polydispersity indices (PDIs) of 0.1-0.15 and controlled drug and magnetite concentrations (Johnson et al. (2003) Phys. Rev. Lett., 91(11); Johnson et al. (2003) Aiche J., 49:2264-82; Johnson et al. (2003) Austr. J. Chem., 56:1021-4; Liu et al. (2007) Phys. Rev. Lett., 98(3); Liu et al. (2008) Chem. Engr. Sci., 63:2829-42). A 4-jet multi-inlet vortex mixer was employed to rapidly combine a solution of the polyester-PEO amphiphilic polymers, ART drugs, and hydrophobically modified magnetite nanoparticles (˜8 nm diameter) with water. The rapid mixing created high supersaturations of the drug and magnetite which led to nucleation and growth of SMART nanoparticles, whereby their size was controlled by the self-assembly of the amphiphilic copolymer onto their surfaces. Conducting these experiments at concentrations of the amphiphilic polymer at least 3× higher than the critical micelle concentration allowed for the PEO of the copolymer to form a repulsive polymer barrier that enabled their colloidal stability. This process is scalable and has been used to produce stable nanoparticles that incorporated drugs, imaging agents, peptides, and targeting ligands with controlled particle size distributions (Ungun et al. (2009) Optics Express 17:80-6; Kumar et al. (2010) Mol. Pharm., 7:291-8; Chen et al. (2009) Nano Letters 9:2218-22; Ansell et al. (2008) J. Med. Chem., 51:3288-96; D'Addio et al. (2011) Adv. Drug Del. Rev., 63:417-26).

### Size and Composition of RTV- and Magnetite-Containing Particles

Flash nanoprecipitation was used to make a series of well-defined particles comprised of magnetite, RTV, and polymers with narrow size distributions. Their polydispersity index values (PDI) as measured by dynamic light scattering typically ranged from 0.10-0.15. Particles of PDLLA (10 k)-PEO (5 k) were made with progressively higher loadings of magnetite (Table 1) and had narrow size distributions.

TABLE 1
Properties of magnetite-containing
particles of PDLLA(10k)-PEO(5k).

| Wt % Magnetite targeted | D (nm) | PDI |
|---|---|---|
| 10% | 88 | 0.15 |
| 20% | 116 | 0.11 |
| 30% | 116 | 0.11 |

Particles comprised of blends of PDLLA (10 k)-PEO (5 k) diblock with PLLA (11 k) homopolymer were made with progressively higher loadings of ritonavir (RTV), an ART drug that is a protease inhibitor. Briefly, the MIVM conditions were: THF stream−11.55 ml/min; water stream (3×)−38.46 ml/min; THF/water=1:10 v/v; concentration of PDLLA (10 k)-PEO (5 k) in the mixer=3 mg/ml; PLLA: PDLLA (10 k)-PEO (5 k)=0.33:1, w/w. For DLS, lyophilized nanoparticles were resuspended in deionized water to 0.1 mg/ml, sonicated in a water bath for 30 minutes, filtered with a 1 μm PTFE filter, and then analyzed by DLS. The RTV concentrations were measured by high pressure liquid chromatography (HPLC). These showed similar sizes and small PDI values (Table 2). It is significant that the RTV loading efficiency increased with RTV targeted loading, reaching a value of 90% and an RTV loading of 45 wt % when the targeted loading was 50 wt % (˜90% drug loading efficiency). This higher efficiency occurred as a result of higher supersaturation values of the RTV in the multi-inlet vortex mixer which led to higher drug nucleation rates. This is consistent with other studies of particle formation using flash nanoprecipitation (Johnson et al. (2003) Austr. J. Chem., 56:1021-4).

TABLE 2
The RTV loading efficiency of SMART particles made by flash
nanoprecipitation increases significantly as the targeted wt
% RTV increases while the polydispersity remains very low.

| | Wt % | Wt % | | Z- | |
|---|---|---|---|---|---|
| | RTV | RTV | D | avg | |
| Polymer | targeted | measured | (nm) | (nm) | PDI |
| PLLA(11k)/PDLLA(10k)- | 0 | — | 125 | 107 | 0.14 |
| PEO(5k) | | | | | |
| PLLA(11k)/PDLLA(10k)- | 20 | 7.6 | 143 | 123 | 0.15 |
| PEO(5k) | | | | | |
| PLLA(11k)/PDLLA(10k)- | 33 | 21.1 | 128 | 107 | 0.15 |
| PEO(5k) | | | | | |
| PLLA(11k)/PDLLA(10k)- | 50 | 45.4 | 131 | 113 | 0.15 |
| PEO(5k) | | | | | |

PDLLA-PEO = poly(DL-lactic acid)-b-poly(ethylene oxide).
PLLA = poly(L-lactic acid) homopolymer.
Numbers in parentheses are molecular weights of blocks (kD).
D is the intensity-average hydrodynamic diameter.

Particles were also made with combinations of RTV, magnetite, and polymers (Table 3). RTV loadings high enough to be therapeutically useful were achieved while the magnetite loadings were also high enough to serve as an effective MRI imaging agent. The magnetite loadings were all within 20% of their targeted values. These results also demonstrate the ability to tune particle size by controlling the polymer chemistry. The first 2 samples, which were made with just PDLLA-PEO diblock copolymers, had diameters in the range 100-115 nm while the latter two samples, which were made with blends of PDLLA-PEO and PLLA homopolymer, are ˜20-30% larger.

TABLE 3
SMART particles containing RTV and magnetite made by flash
nanoprecipitation have narrow size distributions.

| | Wt % | Wt % | D | |
|---|---|---|---|---|
| Polymer | RTV | magnetite | (nm) | PDI |
| PDLLA(10k)-PEO(5k) | 0 | 22.3 | 101 | 0.13 |
| PDLLA(10k)-PEO(5k) | 13.7 | 19.6 | 114 | 0.15 |
| PLLA(11k)/PDLLA(10k)-PEO(5k) | 6.8 | 17.4 | 137 | 0.10 |
| PLLA(11k)/PDLLA(10k)-PEO(5k) | 6.9 | 17.1 | 135 | 0.10 |

PDLLA-PEO = poly(DL-lactic acid)-b-poly(ethylene oxide).
PLLA = poly(L-lactic acid) homopolymer.
Numbers in parentheses are molecular weights of blocks (kD).

### MRI Relaxivity Properties

Another example of well-defined particles shows magnetite nanoparticles clustered in particle cores comprised of PDLLA. The transverse relaxivity (r₂) of these particles was 362 s⁻¹ mM Fe⁻¹ as measured in water at 37° C. and at a field strength of 1.4 Tesla. By comparison, r₂ for a commercially available magnetite-based contrast agent, Feridex™, is 41 s⁻¹ mM Fe⁻¹ measured at 1.5 T and 37° C. (Rohrer et al. (2005) Invest. Radiol., 40:715-24). Moreover, an MTT cytotoxicity study of the particles showed that they were not toxic at concentrations at least as high as 0.5 mg Fe/mL. The transverse relaxivity of magnetite-polymer nanoparticles depends on several factors including particle size, magnetite loading, and the field strength of the MRI measurement (Carroll et al. (2011) Nanotechnol., 22(32)). This is demonstrated by relaxivity measurements conducted on another sample [magnetite (22.2 wt %)/PDLLA (10 k)-PEO (5 k) (77.8 wt %)] in water at 37° C. and at a field strength of 7 Tesla resulted in r₂=217 s⁻¹ mM Fe⁻¹. By comparison, for Feridex™ at those same conditions, r₂=260 s-1 mM Fe⁻¹. Overall, the instant results indicate the SMART particles can readily be used in MRI biodistribution experiments.

### ATV-Containing Particles

Nanoparticles comprising atazanavir (ATV), an ART drug also used as a protease inhibitor, and the PCL-PEO diblock polymer blended with novel PCL-Pluronic-PCL pentablock copolymers were also synthesized. The poly(propylene oxide) or PPO block in the Pluronics copolymer was used to improve the compatibility of the semicrystalline PCL for ATV. These particles were made by precipitation from an organic solution using dialysis to exchange the solvent with water rather than by flash nanoprecipitation. Particles consisting of ATV/magnetite/polymer were made using this approach resulting in loadings of 12 wt % ATV and 11 wt % magnetite and an intensity average particle diameter=265 nm with PDI=0.15. Particles were also made with 30 wt % ATV loading (no magnetite) with an intensity average particle diameter=403 nm and PDI=0.26. Significant increases in both ART and magnetite loading with PDI values less than 0.2 would be achieved using the flash nanoprecipitation process with these PPO-containing copolymers instead of the relatively slow mixing that occurs using the dialysis procedure.

### EXAMPLE 3

Preparation of Magnetite Loaded PLGA Particles with DSPC/mPEG-DSPE or DSPC/DSPE-PEG-Folate Coating

The preparation of the magnetite loaded DSPC/mPEG-DSPE (non-targeted) or DSPC/DSPE-PEG-Folate (targeted) coated PLGA nanoparticle was as follows. The oil phase was prepared by dissolving a weighed amount of PLGA and magnetite (4:1, w/w) in dichloromethane (DCM). The aqueous phase was prepared by hydration of DSPC and mPEG-DSPE films with a molar ratio at 2:1 in water with 10-time volume of DCM. The weight ratio of PLGA and total lipid is 2:1. The oil phase was added to aqueous phase drop-by-drop with constant stirring followed by 60 seconds sonication and a 20 second break under an ice bath, sonication and ice bath procedure was repeated for 2 more cycles. DCM was then removed by placing the container in a fume hood and stirring overnight. The particle suspension was purified by centrifugation at 500×g for 5 minutes then supernatant were collected. The particle was washed to remove excess DSPE and mPEG-DSPE by centrifugation at 50,000×g for 20 minutes, followed by resuspension in phosphate-buffered saline (PBS). The nanoparticle was collected after being washed 3 times as described above.

Magnetite loaded DSPC/DSPE-PEG-Folate and PLGA core particle were prepared by the same protocol, while mPEG-DSPE was substituted with DSPE-PEG-Folate.

Characterization of Magnetite Loaded PLGA Particles with DSPC/mPEG-DSPE or DSPC/DSPE-PEG-Folate Coating

Formulation was diluted in distilled water and particle size and size distribution was measured by dynamic light scattering. The results showed that the average size of the PLGA particles with DSPC/mPEG-DSPE coating is 337 nm while the average size of PLGA particles with DSPC/DSPE-mPEG-Folate coating is 385 nm.

The shape and surface morphology of the SMART particles was investigated by transmission electron microscopy. Samples were prepared from dilutions in distilled water of particle suspensions and dropped onto stubs. After air drying the particles were coated with a thin layer of gold then examined by transmission electron microscopy.

Magnetite loading was accessed by inductively coupled plasma mass spectrometry (ICP-MS). 1 mg lyophilized formulation was weighed out, put into a 10 mL volumetric flask then mixed with 1 mL of 70% nitric acid. The volumetric flask was incubated in 45° C. water bath for 24 hours. Distilled water was added to volumetric flask until volume is 10 mL. 1 mL of solution was used to access the iron content by ICP/MS.

The amount of folate in formulation was determined by UV absorbance at 360 nm and compared against a standard curve of folate prepared in DMSO. Formulation was dissolved in DMSO and sonicated for 5 minutes, and the absorbance was read. Lyophilized formulation was weighted out and dissolved in DMSO and sonicated for 5 minutes, and the absorbance was read. The folate content is 0.29 μg/mg lyophilized PLGA formulation with DSPC/DSPE-mPEG-Folate coating.

A number of publications and patent documents are cited throughout the foregoing specification in order to describe the state of the art to which this invention pertains. The entire disclosure of each of these citations is incorporated by reference herein.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A nanoparticle comprising a hydrophobically modified superparamagnetic particle, a therapeutic agent, and an amphiphilic compound,
wherein the amphiphilic compound forms a layer around a hydrophobic core, and
wherein said hydrophobic core comprises said hydrophobically modified superparamagnetic particle and said therapeutic agent.

2. The nanoparticle of claim 1, wherein said amphiphilic compound is an amphiphilic block copolymer.

3. The nanoparticle of claim 2, wherein at least one hydrophilic block of said amphiphilic block copolymer comprises polyethelene oxide or a polysaccharide.

4. The nanoparticle of claim 2, wherein at least one hydrophobic block of said amphiphilic block copolymer comprises a polyester or polyanhydride.

5. The nanoparticle of claim 1, wherein said amphiphilic compound is a phospholipid.

6. The nanoparticle of claim 5, wherein said phospholipid is linked to a hydrophilic polymer.

7. The nanoparticle of claim 6, wherein said hydrophilic polymer is polyethylene oxide or a polysaccharide.

8. The nanoparticle of claim 1, wherein said hydrophobic core further comprises a hydrophobic polymer.

9. The nanoparticle of claim 8, wherein hydrophobic polymer comprises a polyester or a polyanhydride.

10. The nanoparticle of claim 1, wherein said amphiphilic compound is linked to at least one targeting ligand.

11. The nanoparticle of claim 10, wherein said targeting ligand is a macrophage targeting ligand.

12. The nanoparticle of claim 1, wherein said therapeutic agent is an antimicrobial.

13. The nanoparticle of claim 12, wherein said antimicrobial is an antiretroviral.

14. The nanoparticle of claim 13, wherein said antiretroviral is selected from the group consisting of nucleoside-analog reverse transcriptase inhibitors (NRTIs), non-nucleoside reverse transcriptase inhibitors (NNRTIs), protease inhibitors (PI), viral entry inhibitors, and integrase inhibitors.

15. The nanoparticle of claim 1, wherein said hydrophobically modified superparamagnetic particle is an ultrasmall superparamagnetic iron oxide (USPIO) particle.

16. The nanoparticle of claim 15, wherein said USPIO is coated with oleic acid.

17. The nanoparticle of claim 1, synthesized by flash precipitation.

18. A composition comprising at least one nanoparticle of claim 1 and at least one pharmaceutically acceptable carrier.

19. A method for treating or inhibiting a microbial infection in a subject in need thereof, said method comprising administering to said subject at least one composition of claim 18, wherein the therapeutic agent is an antimicrobial compound.

20. The method of claim 19, wherein said microbial infection is an HIV infection and said antimicrobial compound is am anti-HIV compound.

21. The method of claim 20, further comprising the administration of at least one additional anti-HIV compound.

22. A method for monitoring the pharmacokinetics and/or biodistribution of a therapeutic agent in a subject, said method comprising:
a) administering to said subject at least one nanoparticle of claim 1; and
b) performing at least one magnetic resonance imaging procedure, thereby determining the distribution of the therapeutic agent within the subject.

23. A method of synthesizing the nanoparticle of claim 1, said method comprising performing a flash precipitation wherein an organic solution comprising said hydrophobically modified superparamagnetic particle, therapeutic agent, and amphiphilic compound is mixed with water or an aqueous solution.
